(19) United States
(12) Patent Application Publication
(10) Pub. No.: US 2025/0283051 A1
(43) Pub. Date: Sep. 11, 2025

(54) CELLS DIFFERENTIATED FROM IMMUNOENGINEERED PLURIPOTENT CELLS

(71) Applicant: The Regents of the University of California, Oakland, CA (US)

(72) Inventors: Sonja Schrepfer, Burlingame, CA (US); Tobias Deuse, Burlingame, CA (US)

(73) Assignee: The Regents of the University of California, Oakland, CA (US)

(21) Appl. No.: 19/015,552

(22) Filed: Jan. 9, 2025

Related U.S. Application Data
(62) Division of application No. 17/260,224, filed on Jan. 14, 2021, now abandoned.
(60) Provisional application No. 62/698,965, filed on Jul. 17, 2018.
(60) Provisional application No. 62/698,973, filed on Jul. 17, 2018.
(60) Provisional application No. 62/698,978, filed on Jul. 17, 2018.
(60) Provisional application No. 62/698,981, filed on Jul. 17, 2018.
(60) Provisional application No. 62/698,984, filed on Jul. 17, 2018.

(51) Int. Cl. C12N 5/074; C12N 5/077

(52) U.S. Cl. CPC C12N 5/0696; C12N 5/0657; C12N 2500/38; C12N 2501/115; C12N 2501/165; C12N 2501/235; C12N 2501/33; C12N 2501/385; C12N 2501/415; C12N 2501/602; C12N 2501/603; C12N 2501/604; C12N 2501/606; C12N 2501/608; C12N 2506/11; C12N 2506/1307; C12N 2506/45; C12N 2533/54

(57) ABSTRACT

The invention provides universally acceptable “off-the-shelf” hypoimmunogenic pluripotent cells and differentiated cardiac, endothelial, neuronal, islet, or retinal pigment cells thereof. Such hypoimmune cells are used to treat patients in need thereof. The cells lack major immune antigens that trigger immune responses and are engineered to avoid phagocytic endocytosis.

### I. CROSS REFERENCE TO RELATED APPLICATIONS

This application is a divisional of U.S. application Ser. No. 17/260,224 filed Jan. 14, 2021, which is the U.S. National Stage Application of PCT/US2019/042117 filed Jul. 17, 2019, and claims priority to U.S. Provisional Application Nos. 62/698,965 filed on Jul. 17, 2018, 62/698,973 filed on Jul. 17, 2018, 62/698,978 filed on Jul. 17, 2018, 62/698,981 filed on Jul. 17, 2018, and 62/698,984 filed on Jul. 17, 2018, all incorporated by reference herein in its entirety.

### SEQUENCE LISTING

The instant application contains a Sequence Listing which has been submitted electronically in XML file format and is hereby incorporated by reference in its entirety. Said XML copy, created on Mar. 6, 2025, is named RUC007US2_SL.xml and is 20,794 bytes in size.

### II. FIELD OF THE INVENTION

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g., after myocardial infarction). The propensity for the transplant recipient's immune system to reject allogeneic material, however, greatly reduces the potential efficacy of therapeutics and diminishes the possible positive effects surrounding such treatments.

Thus, the invention provides universally acceptable “off-the-shelf” hypoimmunogenic pluripotent cells and differentiated cardiac, endothelial, neuronal, islet, or retinal pigment cells thereof. Such hypoimmune cells are used to treat patients in need thereof. The cells lack major immune antigens that trigger immune responses and are engineered to avoid phagocytic endocytosis.

### III. BACKGROUND OF THE INVENTION

Regenerative cell therapy is an important potential treatment for regenerating injured organs and tissue. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g. after myocardial infarction). The propensity for the transplant recipient's immune system to reject allogeneic material, however, greatly reduces the potential efficacy of therapeutics and diminishes the possible positive effects surrounding such treatments.

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies are easier from a manufacturing standpoint and allow the generation of well-screened, standardized, high-quality cell products. Because of their allogeneic origin, however, such cell products would undergo rejection. With the reduction or elimination of the cells' antigenicity, universally-acceptable cell products could be produced. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation.

In most cases, however, undifferentiated pluripotent stem cells are avoided in clinical transplant therapies due to their propensity to form teratomas. Rather, such therapies tend to use differentiated cells (e.g. stem cell-derived cardiomyocytes transplanted into the myocardium of patients suffering from heart failure). Clinical applications of such pluripotent cells or tissues would benefit from a “safety feature” that controls the growth and survival of cells after their transplantation.

The art seeks stem cells capable of producing cells that are used to regenerate or replace diseased or deficient cells. Pluripotent stem cells (PSCs) may be used because they rapidly propagate and differentiate into many possible cell types.

To date, preclinical success of PSC-based approaches has only been achieved in immunosuppressed or immunodeficient models, or when the cells are encapsulated and protected from the host's immune system. Systemic immunosuppression as used in allogeneic organ transplantation, however, is not justifiable for regenerative approaches. Immunosuppressive drugs have severe side effects and significantly increase the risk of infections and malignancies.

There is a need in the art for cells produced from hypoimmunogenic pluripotent stem cells that can be used in regenerative medicine.

### IV. SUMMARY OF THE INVENTION

In some aspects, provided herein is an isolated, engineered hypoimmune cardiac, endothelial, neuronal, islet, or retinal pigment cells differentiated from hypoimmune pluripotent stem cells (HIP cells). The HIP cells have, for example, a reduced or eliminated endogenous β-2 microglobulin (B2M) gene activity, reduced or eliminated endogenous class II transactivator (CIITA) gene activity, and increased CD47 expression.

In some embodiments, the HIP cell is a human engineered induced pluripotent stem cell (human engineered iPSC), the B2M gene is human B2M gene, the CIITA gene is human B2M gene, and the increased CD47 expression results from introducing into the cell at least one copy of a human CD47 gene under the control of a promoter. In certain embodiments, the HIP is a mouse engineered induced pluripotent stem cell (mouse engineered iPSC), the B2M gene is mouse B2M gene, the CIITA gene is mouse B2M gene, and the increased CD47 expression results from introducing into the cell at least one copy of a mouse CD47 gene under the control of a promoter. In some instances, the elimination of B2M gene activity results from a Clustered Regularly Interspaced Short Palindromic Repeats (CRISPR)/Cas9 reaction that disrupts both alleles of the B2M gene. In some stances, the elimination of CIITA gene activity results from a CRISPR/Cas9 reaction that disrupts both alleles of the CIITA gene.

In some embodiments, the method further comprises a suicide gene that is activated by a trigger agent that induces the HIP cell to die. In some embodiments, the suicide gene is a herpes simplex virus thymidine kinase (HSV-tk) gene and the trigger agent is ganciclovir. In some instances, the HSV-tk gene encodes a protein comprising at least 90% sequence identity to SEQ ID NO:4. In other instances, the HSV-tk gene encodes a protein comprising the amino acid sequence of SEQ ID NO:4.

In other embodiments, the suicide gene is an Escherichia coli cytosine deaminase (CD) gene and the trigger agent is 5-fluorocytosine (5-FC). In some instances, the CD gene encodes a protein comprising at least 90% sequence identity to SEQ ID NO:5. In other instances, the CD gene encodes a protein comprising the amino acid sequence of SEQ ID NO: 5.

In other embodiments, the suicide gene encodes an inducible caspase 9 protein and the trigger agent is a chemical inducer of dimerization (CID). In some instances, the inducible caspase 9 protein comprises at least 90% sequence identity to SEQ ID NO:6. In other instances, the inducible caspase 9 protein comprises the amino acid sequence of SEQ ID NO: 6. In some cases, the CID is compound AP1903.

In some embodiments, the isolated hypoimmune cardiac cell is selected from the group consisting of a cardiomyocyte, nodal cardiomyocyte, conducting cardiomyocyte, working cardiomyocyte, cardiomyocyte precursor, cardiomyocyte progenitor cell, cardiac stem cell, and cardiac muscle cell.

In some aspects, provided herein is a method of treating a patient suffering from a heart condition or disease. The method comprises administering a composition comprising a therapeutically effective amount of a population of any one of the isolated, engineered hypoimmune cardiac cells described herein. In some embodiments, the composition further comprises a therapeutically effective carrier.

In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

In some aspects, provided herein is a method of producing a population of hypoimmune cardiac cells from a population of hypoimmune pluripotent cells (HIP cells) by in vitro differentiation, wherein endogenous β-2 microglobulin (B2M) gene activity and endogenous class II transactivator (CIITA) gene activity have been eliminated and CD47 expression has been increased in the HIP cells. The method comprises: (a) culturing a population of HIP cells in a culture medium comprising a GSK inhibitor: (b) culturing the population of HIP cells in a culture medium comprising a WNT antagonist to produce a population of pre-cardiac cells; and (c) culturing the population of pre-cardiac cells in a culture medium comprising insulin to produce a population of hypoimmune cardiac cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the WNT antagonist is IWR1, a derivative thereof, or a variant thereof. In some instances, the WNT antagonist is at a concentration ranging from about 2 μM to about 10 μM.

In some embodiments, the method further comprises culturing the population of pre-cardiac cells of step (c) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein. In certain embodiments, the method further comprises culturing the population of hypoimmune cardiac cells of step (d) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In some embodiments, the method further comprises isolating the population of hypoimmune cardiac cells from non-cardiac cells. In other embodiments, the method further comprises cryopreserving the isolated population of hypoimmune cardiac cells.

In some embodiments, the isolated, engineered hypoimmune endothelial cell is selected from the group consisting of a capillary endothelial cell, vascular endothelial cell, aortic endothelial cell, brain endothelial cell, and renal endothelial cell.

In some aspects, provided herein is a method of treating a patient suffering from a vascular condition or disease. In some embodiments, the method comprises administering a composition comprising a therapeutically effective amount of a population of isolated, engineered hypoimmune endothelial cells.

The method comprises administering a composition comprising a therapeutically effective amount of a population of any one of the isolated, engineered hypoimmune endothelial cells described herein. In some embodiments, the composition further comprises a therapeutically effective carrier. In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the vascular condition or disease is selected from the group consisting of, vascular injury, cardiovascular disease, vascular disease, ischemic disease, myocardial infarction, congestive heart failure, hypertension, ischemic tissue injury, limb ischemia, stroke, neuropathy, and cerebrovascular disease.

In some aspects, provided herein is a method of producing a population of hypoimmune endothelial cells from a population of hypoimmunogenic pluripotent stem cells (HIP cells) by in vitro differentiation, wherein endogenous β-2 microglobulin (B2M) gene activity and endogenous class II transactivator (CIITA) gene activity have been eliminated and CD47 expression has been increased in the HIP cells. The method comprises: (a) culturing a population of HIP cells in a first culture medium comprising a GSK inhibitor: (b) culturing the population of HIP cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmune endothelial cells.

In some embodiments, the GSK inhibitor is CHIR-99021, a derivative thereof, or a variant thereof. In some instances, the GSK inhibitor is at a concentration ranging from about 1 μM to about 10 μM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 μM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 μM SB-431542. In certain embodiments, the third culture medium further comprises VEGF and bFGF. In particular instances, the first culture medium and/or the second medium is absent of insulin.

In some embodiments, the method further comprises isolating the population of hypoimmune endothelial cells from non-endothelial cells. In some embodiments, the method further comprises cryopreserving the isolated population of hypoimmune endothelial cells.

In some embodiments, the isolated hypoimmune dopaminergic neuron is selected from the group consisting of a neuronal stem cell, neuronal progenitor cell, immature dopaminergic neuron, and mature dopaminergic neuron.

In some aspects, provided herein is a method of treating a patient suffering from a neurodegenerative disease or condition. In some embodiments, the method comprises administering a composition comprising a therapeutically effective amount of a population of any one of the isolated hypoimmune dopaminergic neurons. In some embodiments, the composition further comprises a therapeutically effective carrier. In some embodiments, the population of the isolated hypoimmune dopaminergic neurons is on a biodegradable scaffold. The administration may comprise transplantation or injection. In some embodiments, the neurodegenerative disease or condition is selected from the group consisting of Parkinson's disease, Huntington disease, and multiple sclerosis.

In some aspects, provided herein is a method of producing a population of hypoimmune dopaminergic neurons from a population of hypoimmune induced pluripotent stem cells (HIP cells) by in vitro differentiation, wherein endogenous β-2 microglobulin (B2M) gene activity and endogenous class II transactivator (CIITA) gene activity have been eliminated and CD47 expression has been increased in the HIP cells. In some embodiments, the method comprises (a) culturing the population of HIP cells in a first culture medium comprising one or more factors selected from the group consisting of sonic hedgehog (SHH), BDNF, EGF, bFGF, FGF8, WNTI, retinoic acid, a GSK3β inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons.

In some embodiments, the GSKβ inhibitor is CHIR-99021, a derivative thereof, or a variant thereof. In some instances, the GSKβ inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method further comprises culturing the population of immature dopaminergic neurons of step (a) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In some embodiments, the method also comprises isolating the population of hypoimmune dopaminergic neurons from non-dopaminergic neurons.

In some embodiments, the isolated hypoimmune pancreatic islet cell is selected from the group consisting of a pancreatic islet progenitor cell, immature pancreatic islet cell, and mature pancreatic islet cell.

In some aspects, provided herein is a method of treating a patient suffering from diabetes. The method comprises administering a composition comprising a therapeutically effective amount of a population of any one of the isolated hypoimmune pancreatic islet cells described herein. In some embodiments, the composition further comprises a therapeutically effective carrier. In some embodiments, the population of the isolated hypoimmune pancreatic islet cells is on a biodegradable scaffold. In some instances, the administration comprises transplantation or injection.

In some aspects, provided herein is a method of producing a population of hypoimmune pancreatic islet cells from a population of hypoimmune pluripotent cells (HIP cells) by in vitro differentiation, wherein endogenous β-2 microglobulin (B2M) gene activity and endogenous class II transactivator (CIITA) gene activity have been eliminated and CD47 expression has been increased in the HIP cells. The method comprises: (a) culturing the population of HIP cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-β (TGFβ) superfamily, bone morphogenic protein-2 (BMP2), bone morphogenic protein-7 (BMP7), a GSK3ß inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method further comprises culturing the population of immature pancreatic islet cells of step (a) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In particular embodiments, the method further comprises culturing the population of pancreatic islet cells of step (b) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In some embodiments, the method also comprises isolating the population of hypoimmune pancreatic islet cells from non-pancreatic islet cells. In some embodiments, the method further comprises cryopreserving the isolated population of hypoimmune pancreatic islet cells.

In some embodiments, the isolated hypoimmune RPE cell is selected from the group consisting of a RPE progenitor cell, immature RPE cell, mature RPE cell, and functional RPE cell.

In some aspects, provided herein is a method of treating a patient suffering from an ocular condition. The method comprises administering a composition comprising a therapeutically effective amount of a population of any one of a population of the isolated hypoimmune RPE cells described herein. In some embodiments, the composition further comprises a therapeutically effective carrier. In some embodiments, the population of the isolated hypoimmune RPE cells is on a biodegradable scaffold. In some embodiments, the administration comprises transplantation or injection to the patient's retina. In some embodiments, the ocular condition is selected from the group consisting of wet macular degeneration, dry macular degeneration, juvenile macular degeneration, Leber's Congenital Ameurosis, retinitis pigmentosa, and retinal detachment.

In some aspects, provided herein is a method of producing a population of hypoimmune retinal pigmented epithelium (RPE) cells from a population of hypoimmune pluripotent cells (HIP cells) by in vitro differentiation, wherein endogenous β-2 microglobulin (B2M) gene activity and endogenous class II transactivator (CIITA) gene activity have been eliminated and CD47 expression has been increased in the HIP cells. The method comprises: (a) culturing the population of HIP cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmune RPE cells.

In some embodiments, the ALK inhibitor is SB-431542, a derivative thereof, or a variant thereof. In some instances, the ALK inhibitor is at a concentration ranging from about 2 μM to about 10 μM. In some embodiments, the ROCK inhibitor is Y-27632, a derivative thereof, or a variant thereof. In some instances, the ROCK inhibitor is at a concentration ranging from about 1 μM to about 10 μM.

In some embodiments, the first culture medium and/or second culture medium are absent of animal serum.

In some embodiments, the method further comprises culturing the population of pre-RPE cells of step (a) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In some embodiments, the method further comprises culturing the population of RPE cells of step (b) in a culture medium comprising a trigger agent if the HIP cells comprise a suicide gene, wherein the trigger agent is ganciclovir if the suicide gene is a herpes simplex virus thymidine kinase (HSV-tk) gene, the trigger agent is 5-fluorocytosine (5-FC) if the suicide gene is an Escherichia coli cytosine deaminase (CD) gene, or the trigger agent is a chemical inducer of dimerization (CID) if the suicide gene encodes an inducible caspase 9 protein.

In some embodiments, the method further comprises isolating the population of hypoimmune RPE cells from non-RPE cells. In some embodiments, the method further comprises cryopreserving the isolated population of hypoimmune RPE cells.

### V. BRIEF DESCRIPTION OF THE DRAWINGS

FIG. 1 shows Elispot results of mouse B2m−/−Ciita−/−CD47 tg iPSCs incubated with mouse NK cells (approximately 95% NK cells and 5% macrophages).

FIG. 2 shows Elispot results of human B2M−/−CIITA−/−CD47 tg iPSCs incubated with human NK cells (approximately 95% NK cells and 5% macrophages).

FIG. 3 shows Elispot results of mouse B2m−/−Ciita−/−CD47 tg iPSCs incubated with human NK cells (approximately 95% NK cells and 5% macrophages).

FIG. 4 shows Elispot results of human B2M−/−CIITA−/−CD47 tg iPSCs incubated with mouse NK cells (approximately 95% NK cells and 5% macrophages).

FIG. 5 shows phagocytosis assay results of firefly luciferase labeled human B2M−/−CIITA−/−CD47 tg iPSCs co-cultured with human macrophages.

FIG. 6 shows phagocytosis assay results of firefly luciferase labeled mouse B2m−/−Ciita−/−CD47 tg iPSCs co-cultured with mouse macrophages.

FIG. 7 shows phagocytosis assay results of firefly luciferase labeled human B2M−/−CIITA−/−CD47 tg iPSCs co-cultured with mouse macrophages.

FIG. 8 shows phagocytosis assay results of firefly luciferase labeled mouse B2m−/−Ciita−/−CD47 tg iPSCs co-cultured with human macrophages.

FIG. 9 provides a diagram of the differentiation method.

FIG. 10 shows human iPSCs cultured on Matrigel™ immediately before starting the differentiation (100× magnification).

FIG. 11 shows cells on differentiation day 2 before media change (100× magnification).

FIG. 12 shows cells on differentiation day 3 before media change (100× magnification).

FIG. 13 shows cells on differentiation day 5 before media change (100× magnification).

FIG. 14 shows cells on differentiation day 7 before media change (100× magnification).

FIG. 15 shows cells on differentiation day 9 before media change (100× magnification).

FIG. 16: HIP-CM cells were differentiated and enriched as shown by rtPCR.

FIG. 17 shows that the hiCMs were not rejected and did not migrate into other organs 28 days post-transplantation.

FIG. 18 shows histopathology and trichrome staining of recipient hearts 28 days after myocardial infarction. The infarct size in allogeneic recipients of hiCMs was significantly reduced, as well as was the size of the left ventricle.

FIG. 19A shows detailed PV-loop analysis demonstrating a significant improvement of left-ventricular parameters.

FIG. 19B shows that the hiCMs restored heart function as measured by ejection fraction (EF, ratio of the volume of blood ejected from the ventricle per beat to the volume of blood in that ventricle at the end of diastole) and stroke volume (SV, the volume of blood ejected by a ventricle in a single contraction).

FIG. 19C shows that the hiCMs restored heart function as measured by ventricular stroke work (SW, the work performed by the left ventricle to eject the stroke volume into the aorta) and cardiac output (CO, the amount of blood pumped by the ventricle in unit time).

FIG. 20 shows results of analyses in which wild-type or B2M−/−CIITA−/−CD47 tg hiCMs were transplanted intramuscularly into allogeneic humanized mice.

FIG. 21 shows that the hypoimmune hiCM cells also survived following transplantation after myocardial infarction.

FIG. 22 shows mouse hypo-IPSC colonies on MEFs before splitting (100× magnification).

FIG. 23 shows ESCmouse hypo-IPSC on gelatin immediately before starting differentiation (100× magnification).

FIG. 24 shows cells on day 2 of differentiation (100× magnification) before the differentiation media was changed from 5 μM CHIR to 2 μM CHIR.

FIG. 25 shows cells on day 4 of EC differentiation (100× magnification) before the media was changed.

FIG. 26 shows EC cells on day 7 of differentiation (100× magnification).

FIG. 27 shows cells after day 12-day 14 and prior to MACS sorting (100× magnification).

FIG. 28 shows rtPCR that demonstrates that the EC cells from both the miPSCs and the HIP cells showed a differentiated gene expression profile, including VE-cadherin expression, where the parent cells did not.

FIG. 29 shows bioluminescence analyses of wild-type and hypoimmune induced endothelial cells transplanted in a hindlimb ischemia mouse model (after removal of the A. femoralis). BLI values of all animals were plotted. Very immunogeneic wt mECs were rejected within 15 days showing declining BLI signals over time while the B2M−/−CIITA−/−CD47 tg grafts all survived.

FIG. 30. The inhibitory effect of Cd47 over-expression on NK cell killing was assessed. IFN-γ Elispots with NK cells were performed with miECs derived from B2m^{-/-} Ciita^{-/-} miPSC or B2m^{-/-}Ciita^{-/-} Cd47 tg miPSC. Only derivatives from B2m^{-/-}Ciita^{-/-} miPSC were susceptible for NK cell killing.

FIG. 31. shows that B2M^{-/-}CIITA^{-/-} CD47 tg hiPSCs were successfully differentiated into corresponding hiEC derivatives. rtPCR shows that all derivatives lost pluripotency gene expression.

FIG. 32. Wild-type or B2M−/−CIITA−/−CD47 tg hiEC grafts were injected into allogeneic humanized NSG-SGM3 mice. IFN-γ Elispots were performed after 5 days. The hypoimmune cells did not elicit IFN-γ responses but the wild-type did.

### VI. DETAILED DESCRIPTION OF THE INVENTION

### A. Introduction

The invention provides for the generation of cardiac cells derived from (differentiated from HypoImmunogenic Pluripotent (HIP) cells, and ultimately transplantation into patients in need thereof.

As described in PCT/US18/13688, hypoimmunogenic pluripotent (HIP) cells lack major immune antigens that can trigger immune responses and are engineered to avoid phagocytosis. This allows the derivation of “off-the-shelf” cell products for generating specific tissues and organs. The benefit of being able to use human allogeneic HIP cell derivatives in human patients results in significant benefits, including the ability to avoid long-term adjunct immunosuppressive therapy and drug use generally seen in allogeneic transplantations. It also provides significant cost savings as cell therapies can be used without requiring individual treatments for each patient.

It has been shown that cell products generated from autologous cell sources may become subject to immune rejection with few or even one single antigenic mutation. Thus, autologous cell products are not inherently non-immunogenic. Also, as cell engineering and quality control is very labor- and cost-intensive, autologous cells may not be readily available for acute treatment options. Thus, there is a need in the art for a universal cell source such as HIP cells that can be differentiated into numerous types of universally-acceptable cells to be used to treat various human diseases.

This application is related to International Application No. PCT/US18/13688, filed on Jan. 14, 2018 and U.S. Provisional Application No. 62/445,969, filed Jan. 13, 2017, the disclosures in their entirety are herein incorporated by reference, in particular, the examples, figures, figure descriptions, and descriptions of producing hypoimmunogenic pluripotent stem cells and differentiating such cells into other cell types.

### B. Definitions

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al. New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009): Huangfu et al., Nature Biotechnol. 26 (7): 795 (2008): Woltjen et al., Nature 458 (7239): 766-770 (2009); and Zhou et al., Cell Stem Cell 8:381-384 (2009): each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic pluripotent cell,” “hypoimmune pluripotent cell.” or “HIP cell” herein is meant a pluripotent cell that retains its pluripotent characteristics and yet gives rise to a reduced immunological rejection response when transferred into an allogeneic host. In preferred embodiments, HIP cells do not give rise to an immune response. Thus. “hypo-immunogenic” or “hypoimmune” refers to a significantly reduced or eliminated immune response when compared to the immune response of a parental (i.e. “wild-type” or “wt”) cell prior to immunoengineering as outlined herein. In many cases, the HIP cells are immunologically silent and yet retain pluripotent capabilities. Assays for HIP characteristics are outlined below.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM. HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below; this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

“β-2 microglobulin” or “β2M” or “B2M” protein refers to the human β2M protein that has the amino acid and nucleic acid sequences shown below: the human gene has accession number NC_000015.10:44711487-44718159.

“CD47 protein” protein refers to the human CD47 protein that has the amino acid and nucleic acid sequences shown below: the human gene has accession number NC_000003.12:108043094-108094200.

“CIITA protein” protein refers to the human CIITA protein that has the amino acid and nucleic acid sequences shown below: the human gene has accession number NC_000016.10:10866208-10941562.

By “wild type” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

By “syngeneic” herein refers to the genetic similarity or identity of a host organism and a cellular transplant where there is immunological compatibility: e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “CIITA−/−” herein is meant that a diploid cell has had the CIITA gene inactivated in both chromosomes. As described herein, this can be done in a variety of ways.

By “CD47 tg” (standing for “transgene”) or “CD47+”) herein is meant that the host cell expresses CD47, in some cases by having at least one additional copy of the CD47 gene.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7. Oct-8, Oct-9, and Oct-11. Oct3/4 (referred to herein as “Oct4”) contains the POU domain, a 150 amino acid sequence conserved among Pit-1, Oct-1, Oct-2, and uric-86. (See, Ryan, A. K. & Rosenfeld. M. G., Genes Dev. 11:1207-1225 (1997), incorporated herein by reference in its entirety.) In some embodiments, variants have at least 85%, 90%, or 95% amino acid sequence identity across their whole sequence compared to a naturally occurring Oct polypeptide family member such as to those listed above or such as listed in Genbank accession number NP-002692.2 (human Oct4) or NP-038661.1 (mouse Oct4). Oct polypeptides (e.g., Oct3/4 or October 4) can be from human, mouse, rat, bovine, porcine, or other animals. Generally, the same species of protein will be used with the species of cells being manipulated. The Oct polypeptide(s) can be a pluripotency factor that can help induce multipotency in non-pluripotent cells.

A “Klf polypeptide” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. (See, Dang. D. T., Pevsner, J. & Yang. V. W., Cell Biol. 32:1103-1121 (2000), incorporated by reference herein in its entirety.) Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Klf polypeptide family member such as to those listed above or such as listed in GenBank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. The Klf polypeptide(s) can be a pluripotency factor. The expression of the Klf4 gene or polypeptide can help induce multipotency in a starting cell or a population of starting cells.

A “Myc polypeptide” refers to any of the naturally-occurring members of the Myc family. (See. e.g., Adhikary, S. & Eilers, M., Nat. Rev. Mol. Cell Biol. 6:635-645 (2005), incorporated by reference herein in its entirety.) It also includes variants that maintain similar transcription factor activity when compared to the closest related naturally occurring family member (i.e. within at least 50%, 80%, or 90% activity). It further includes polypeptides comprising at least the DNA-binding domain of a naturally occurring family member, and can further comprise a transcriptional activation domain. Exemplary Myc polypeptides include, e.g., c-Myc. N-Myc and L-Myc. In some embodiments, variants have at least 85%, 90%, or 95% amino acid sequence identity across their whole sequence compared to a naturally occurring Myc polypeptide family member, such as to those listed above or such as listed in GenBank accession number CAA25015 (human Myc). Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor.

A “Sox polypeptide” refers to any of the naturally-occurring members of the SRY-related HMG-box (Sox) transcription factors, characterized by the presence of the high-mobility group (HMG) domain, or variants thereof that maintain similar transcription factor activity when compared to the closest related naturally occurring family member (i.e. within at least 50%, 80%, or 90% activity). It also includes polypeptides comprising at least the DNA-binding domain of the naturally occurring family member, and can further comprise a transcriptional activation domain. (See. e.g., Dang, D. T. et al., Int. J. Biochem. Cell Biol. 32:1103-1121 (2000), incorporated by reference herein in its entirety.) Exemplary Sox polypeptides include, e.g., Sox1, Sox-2, Sox3, Sox4, Sox5, Sox6, Sox7, Sox8, Sox9, Sox10, Sox11, Sox12, Sox13, Sox 14, Sox15, Sox17, Sox18, Sox-21, and Sox30. Sox1 has been shown to yield iPS cells with a similar efficiency as Sox2, and genes Sox3, Sox 15, and Sox 18 have also been shown to generate iPS cells, although with somewhat less efficiency than Sox2. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Sox polypeptide family member such as to those listed above or such as listed in GenBank accession number CAA83435 (human Sox2). Sox polypeptides (e.g., Sox1, Sox2, Sox3, Sox 15, or Sox18) can be from human, mouse, rat, bovine, porcine, or other animals. Generally, the same species of protein will be used with the species of cells being manipulated. The Sox polypeptide(s) can be a pluripotency factor. As discussed herein, SOX2 proteins find particular use in the generation of iPSCs.

By “differentiated hypo-immunogenic pluripotent cells” or “differentiated HIP cells” or “dHIP cells” herein is meant iPS cells that have been engineered to possess hypoimmunogenicity (e.g. by the knock out of B2M and CIITA and the knock in of CD47) and then are differentiated into a cell type for ultimate transplantation into subjects. Thus, for example HIP cells can be differentiated into hepatocytes (“dHIP hepatocytes”), into beta-like pancreatic cells or islet organoids (“dHIP beta cells”), into endothelial cells (“dHIP endothelial cells”), etc.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity.” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment. homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting. generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al. Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing. while shorter probes need lower temperatures.

“Stringent conditions” or “high stringency conditions”, as defined herein, can be identified by those that: (1) employ low ionic strength and high temperature for washing. for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with ( ) 1% bovine serum albumin/0.1% Ficoll/0.1% polyvinylpyrrolidone/50) Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a CD47, HSVtk, EC-CD, or iCasp9 variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell: e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. HYPOIMMUNOGENIC PLURIPOTENT CELLS

The invention provides compositions and methodologies for generating mouse and human HIP cells, starting with wild type cells, rendering them pluripotent (e.g. making induced pluripotent stem cells, or iPSCs), then generating HIP cells from the iPSC population.

Provided herein is a hypo-immunogenic pluripotent (HIP) stem cell comprising: one or more alterations that inactivate both alleles of an endogeneous B2M gene; one or more alterations that inactivate both alleles of an endogenous CIITA gene; and one or more alterations causing an increased expression of a CD47 gene in the human HIP stem cell: wherein the human HIP stem cell elicits a first Natural Killer (NK) cell response that is lower than a second NK cell response elicited by an induced Pluripotent Stem Cell (iPSC) that comprises said B2M and CIITA alterations but does not comprise said increased CD47 gene expression, and wherein the first and second NK cell responses are measured by determining the IFN-γ levels from NK cells incubated in vitro with either of the human HIP or iPSC that comprise the B2M and CIITA alterations but does not comprise the increased CD47 gene expression. The HIP stem cell can be a murine HIP stem cell. In some embodiments, the HIP stem cell is a human HIP stem cell.

The hypoimmunogenic pluripotent cell can be less susceptible to rejection when transplanted into a subject as a result of the reduced HLA-I function, the reduced HLA-II function, and reduced susceptibility to NK cell killing.

In some embodiments, the hypoimmunogenic pluripotent cell has reduced or lacks β-2 microglobulin protein expression. In a preferred embodiment, a gene encoding the β-2 microglobulin protein is eliminated or knocked out. In a more preferred embodiment, the β-2 microglobulin protein has at least 90% (e.g., 91%, 91%, 92%, 93%, 94%, 95%, 96%, 97%, 98%, 99%, or 100%) sequence identity to SEQ ID NO:1. In a more preferred embodiment, the β-2 microglobulin protein has the sequence of SEQ ID NO:1.

In some embodiments, the HLA-I function is reduced by a reduction in HLA-A protein expression. In a preferred embodiment, a gene encoding the HLA-A protein is eliminated or knocked out. In some embodiments, the HLA-I function is reduced by a reduction in HLA-B protein expression. In a preferred embodiment, a gene encoding the HLA-B protein is eliminated or knocked out. In some embodiments, the HLA-I function is reduced by a reduction in HLA-C protein expression. In a preferred embodiment, a gene encoding the HLA-C protein is eliminated or knocked out. In another embodiment, the hypoimmunogenic pluripotent cells do not comprise an HLA-I function.

In some embodiments, the hypoimmunogenic pluripotent cell has reduced or lacks CIITA protein expression. In a preferred embodiment, a gene encoding the CIITA protein is eliminated or knocked out. In a more preferred embodiment, the CIITA protein has at least 90% (e.g., 91%, 91%, 92%, 93%, 94%, 95%, 96%, 97%, 98%, 99%, or 100%) sequence identity to SEQ ID NO:2. In a more preferred embodiment, the CIITA protein has the sequence of SEQ ID NO:2.

In some embodiments, the HLA-II function is reduced by a reduction in HLA-DP protein expression. In a preferred embodiment, a gene encoding the HLA-DP protein is eliminated or knocked out. In some embodiments, the HLA-II function is reduced by a reduction in HLA-DR protein expression. In a preferred embodiment, a gene encoding the HLA-DR protein is eliminated or knocked out. In some embodiments, the HLA-II function is reduced by a reduction in HLA-DQ protein expression. In a preferred embodiment, a gene encoding the HLA-DQ protein is eliminated or knocked out. The invention provides hypoimmunogenic pluripotent cells that do not comprise an HLA-II function.

The invention provides hypoimmunogenic pluripotent cells with a reduced susceptibility to macrophage phagocytosis or NK cell killing. The reduced susceptibility is caused by the increased expression of a CD47 protein. In some embodiments, the increased CD47 expression results from a modification to an endogenous CD47 gene locus. In other embodiments, the increased CD47 expression results from a CD47 transgene. In a preferred embodiment, the CD47 protein has at least 90% (e.g., 91%, 91%, 92%, 93%, 94%, 95%, 96%, 97%, 98%, 99%, or 100%) sequence identity to SEQ ID NO:3. In a more preferred embodiment, the CD47 protein has the sequence of SEQ ID NO:3.

In another embodiment of the method, the increased expression of a protein that reduces the susceptibility of the pluripotent cell to macrophage phagocytosis results from a modification to an endogenous gene locus. In a preferred embodiment, the endogenous gene locus encodes a CD47 protein. In another embodiment, the increased protein expression results from the expression of a transgene. In a preferred embodiment, the transgene encodes a CD47 protein. In a more preferred embodiment, the CD47 protein has at least 90% (e.g., 91%, 91%, 92%, 93%, 94%, 95%, 96%, 97%, 98%, 99%, or 100%) sequence identity to SEQ ID NO: 3. In a more preferred embodiment, the CD47 protein has the sequence of SEQ ID NO: 3.

In some embodiments, the level of CD47 protein in the HIP cells is higher than the level in a corresponding pluripotent stem cell, e.g., embryonic stem cell or induced pluripotent stem cell. In some instances, the level of murine CD47 protein in the murine HIP cells is higher (e.g., at least 0.5-times higher, at least 1.0-times higher, at least 1.5-times higher, at least 2-times higher, at least 3-times higher, at least 4-times higher, at least 5-times higher, at least 6-times higher, at least 7-times higher, at least 8-times higher, at least 8-times higher, or more) than the level in a corresponding murine pluripotent stem cell. In certain instances, the level of human CD47 protein in the human HIP cells is higher (e.g., at least 0.5-times higher, at least 1.0-times higher, at least 1.5-times higher, at least 2-times higher, at least 3-times higher, at least 4-times higher, at least 5-times higher, at least 6-times higher, at least 7-times higher, at least 8-times higher, at least 8-times higher, or more) than the level in a corresponding human pluripotent stem cell.

Another embodiment of the method further comprises expressing a suicide gene that is activated by a trigger that causes the hypoimmunogenic pluripotent or differentiated progeny cell to die. In a preferred embodiment, the suicide gene is a herpes simplex virus thymidine kinase gene (HSV-tk) and the trigger is ganciclovir. In a more preferred embodiment, the HSV-tk gene encodes a protein having at least 90% (e.g., 91%, 91%, 92%, 93%, 94%, 95%, 96%, 97%, 98%, 99%, or 100%) sequence identity to SEQ ID NO: 4. In a more preferred embodiment, the HSV-tk gene encodes a protein having the sequence of SEQ ID NO:4.

In another embodiment of the method, the suicide gene is an Escherichia coli cytosine deaminase gene (EC-CD) and the trigger is 5-fluorocytosine (5-FC). In a preferred embodiment, the EC-CD gene encodes a protein having at least 90% (e.g., 91%, 91%, 92%, 93%, 94%, 95%, 96%, 97%, 98%, 99%, or 100%) sequence identity to SEQ ID NO:5. In a more preferred embodiment, the EC-CD gene encodes a protein having the sequence of SEQ ID NO: 5.

In another embodiment of the method, the suicide gene encodes an inducible Caspase protein and the trigger is a specific chemical inducer of dimerization (CID). In a preferred embodiment of the method, the gene encodes an inducible caspase protein comprising at least 90% (e.g., 91%, 91%, 92%, 93%, 94%, 95%, 96%, 97%, 98%, 99%, or 100%) sequence identity to SEQ ID NO:6. In a more preferred embodiment, the gene encodes an inducible caspase protein comprising the sequence of SEQ ID NO:6. In a more preferred embodiment, the CID is AP1903.

### A. Methodologies for Genetic Alterations

The invention includes methods of modifying nucleic acid sequences within cells or in cell-free conditions to generate both pluripotent cells and HIP cells. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the pluripotent cells of the invention, as well as the engineering of the iPSCs to become hypo-immunogenic as outlined herein.

In general, these techniques can be used individually or in combination. For example, in the generation of the HIP cells, CRISPR may be used to reduce the expression of active B2M and/or CIITA protein in the engineered cells, with viral techniques (e.g. lentivirus) to knock in the CD47 functionality. Also, as will be appreciated by those in the art, although one embodiment sequentially utilizes a CRISPR step to knock out B2M, followed by a CRISPR step to knock out CIITA with a final step of a lentivirus to knock in the CD47 functionality, these genes can be manipulated in different orders using different technologies.

As is discussed more fully below, transient expression of reprogramming genes is generally done to generate/induce pluripotent stem cells.

a. CRISPR Technologies

In one embodiment, the cells are manipulated using clustered regularly interspaced short palindromic repeats)/Cas (“CRISPR”) technologies as is known in the art. CRISPR can be used to generate the starting iPSCs or to generate the HIP cells from the iPSCs. There are a large number of techniques based on CRISPR, see for example Doudna and Charpentier, Science doi: 10.1126/science. 1258096, hereby incorporated by reference. CRISPR techniques and kits are sold commercially.

b. TALEN Technologies

In some embodiments, the HIP cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

d. Viral Based Technologies

There are a wide variety of viral techniques that can be used to generate the HIP cells of the invention (as well as for the original generation of the iPSCs), including, but not limited to, the use of retroviral vectors, lentiviral vectors, adenovirus vectors and Sendai viral vectors. Episomal vectors used in the generation of iPSCs are described below.

e. Downregulation of Genes Using Interfering RNA

In other embodiments, genes that encode proteins used in HLA molecules are downregulated by RNAi technologies. RNA interference (RNAi) is a process where RNA molecules inhibit gene expression often by causing specific mRNA molecules to degrade. Two types of RNA molecules-microRNA (miRNA) and small interfering RNA (siRNA)—are central to RNA interference. They bind to the target mRNA molecules and either increase or decrease their activity. RNAi helps cells defend against parasitic nucleic acids such as those from viruses and transposons. RNAi also influences development.

sdRNA molecules are a class of asymmetric siRNAs comprising a guide (antisense) strand of 19-21 bases. They contain a 5′ phosphate, 2′Ome or 2′F modified pyrimidines, and six phosphotioates at the 3′ positions. They also contain a sense strand containing 3′ conjugated sterol moieties, 2 phosphotioates at the 3′ position, and 2′Ome modified pyrimidines. Both strands contain 2′ Ome purines with continuous stretches of unmodified purines not exceeding a length of 3. sdRNA is disclosed in U.S. Pat. No. 8,796,443, incorporated herein by reference in its entirety.

For all of these technologies, well known recombinant techniques are used, to generate recombinant nucleic acids as outlined herein. In certain embodiments, the recombinant nucleic acids (either than encode a desired polypeptide, e.g. CD47, or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In some embodiments, the elongation factor 1-alpha promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273:113-120 (1978). The immediate early promoter of the human cytomegalovirus is conveniently obtained as a HindIII E restriction fragment. Greenaway, P. J. et al., Gene 18:355-360 (1982). The foregoing references are incorporated by reference in their entirety.

### B. Generation of Pluripotent Cells

The invention provides methods of producing non-immunogenic pluripotent cells from pluripotent cells. Thus, the first step is to provide the pluripotent stem cells.

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7 (1): 116-125 (2015) for a review, and Lakshmipathy and Vermuri, editors, Methods in Molecular Biology: Pluripotent Stem Cells. Methods and Protocols. Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT: SOX2, OCT4 (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see. e.g., the GibcoR) Episomal hiPSC line, K18945, which is a zero footprint, viral-integration-free human iPSC cell line (see also Burridge et al, 2011, supra).

In general, as is known in the art, iPSCs are made from non-pluripotent cells such as CD34+ cord blood cells, fibroblasts, etc., by transiently expressing the reprogramming factors as described herein.

For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015 January 28:5:8081 (doi: 10.1038/srep08081), hereby incorporated by reference in its entirety and specifically for the methods and reagents for the generation of the miPSCs. See also. e.g., Burridge et al., PLoS One, 2011 6 (4): 18293, hereby incorporated by reference in its entirety and specifically for the methods outlined therein.

In some cases, the pluripotency of the cells is measured or confirmed as outlined herein, for example by assaying for reprogramming factors as is generally shown in PCT/US18/13688 or by conducting differentiation reactions as outlined herein and in the Examples.

### C. Generation of Hypo-Immunogenic Pluripotent Cells

The present invention is directed to the generation, manipulation, growth and transplantation of hypo-immunogenic cells into a patient as defined herein. The generation of HIP cells from pluripotent cells is done with as few as three genetic changes, resulting in minimal disruption of cellular activity but conferring immunosilencing to the cells.

As discussed herein, one embodiment utilizes a reduction or elimination in the protein activity of MHC I and II (HLA I and II when the cells are human). This can be done by altering genes encoding their component. In one embodiment, the coding region or regulatory sequences of the gene are disrupted using CRISPR. In another embodiment, gene translation is reduced using interfering RNA technologies. The third change is a change in a gene that regulates susceptibility to macrophage phagocytosis, such as CD47, and this is generally a “knock in” of a gene using viral technologies.

Additional descriptions of hypoimmune pluripotent cells (HIP cells) can be found in International Application No. PCT/US18/13688, filed on Jan. 14, 2018 and U.S. Provisional Application No. 62/445,969, filed Jan. 13, 2017, the disclosures in their entirety are herein incorporated by reference, in particular, the examples, figures, figure descriptions, and descriptions of producing hypoimmunogenic pluripotent stem cells and differentiating such cells into other cell types.

In some cases, where CRISPR is being used for the genetic modifications, hiPSC cells that contain a Cas9 construct that enable high efficiency editing of the cell line can be used; see. e.g., the Human Episomal Cas9 iPSC cell line, A33124, from Life Technologies.

### 1. HLA-I Reduction

The HIP cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the pluripotent cells can be measured using techniques known in the art and as described below: for example, FACS techniques using labeled antibodies that bind the HLA complex: for example, using commercially available HLA-A,B,C antibodies that bind to the alpha chain of the human major histocompatibility HLA Class I antigens.

### B2M Alteration

In one embodiment, the reduction in HLA-I activity is done by disrupting the expression of the β-2 microglobulin gene in the pluripotent stem cell, the human sequence of which is disclosed herein. This alteration is generally referred to herein as a gene “knock out”, and in the HIP cells of the invention it is done on both alleles in the host cell. Generally, the techniques to do both disruptions is the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected iPSC cultures are dissociated to single cells. Single cells are expanded to full-size colonies and tested for CRISPR edit by screening for presence of aberrant sequence from the CRISPR cleavage site. Clones with deletions in both alleles are picked. Such clones did not express B2M as demonstrated by PCR and did not express HLA-I as demonstrated by FACS analysis (see examples 1 and 6, for example).

Assays to test whether the B2M gene has been inactivated are known and described herein. In one embodiment, the assay is a Western blot of cells lysates probed with antibodies to the B2M protein. In another embodiment, recombinase polymerase amplification (RPA) or reverse transcriptase polymerase chain reactions (RT-PCR) confirm the presence of the inactivating alteration.

In addition, the cells can be tested to confirm that the HLA I complex is not expressed on the cell surface. This may be assayed by FACS analysis using antibodies to one or more HLA cell surface components as discussed above.

It is noteworthy that others have had poor results when trying to silence the B2M genes at both alleles. See. e.g. Gornalusse et al., Nature Biotech. Doi/10.1038/nbt.3860).

### 2. HLA-II Reduction

In addition to a reduction in HLA I, the HIP cells of the invention also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, RPA techniques, RT-PCR techniques, etc.

### CIITA Alteration

In one embodiment, the reduction in HLA-II activity is done by disrupting the expression of the CIITA gene in the pluripotent stem cell, the human sequence of which is shown herein. This alteration is generally referred to herein as a gene “knock out”, and in the HIP cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, recombinase polymerase amplification (RPA) or reverse transcriptase polymerase chain reactions (RT-PCR) confirm the presence of the inactivating alteration.

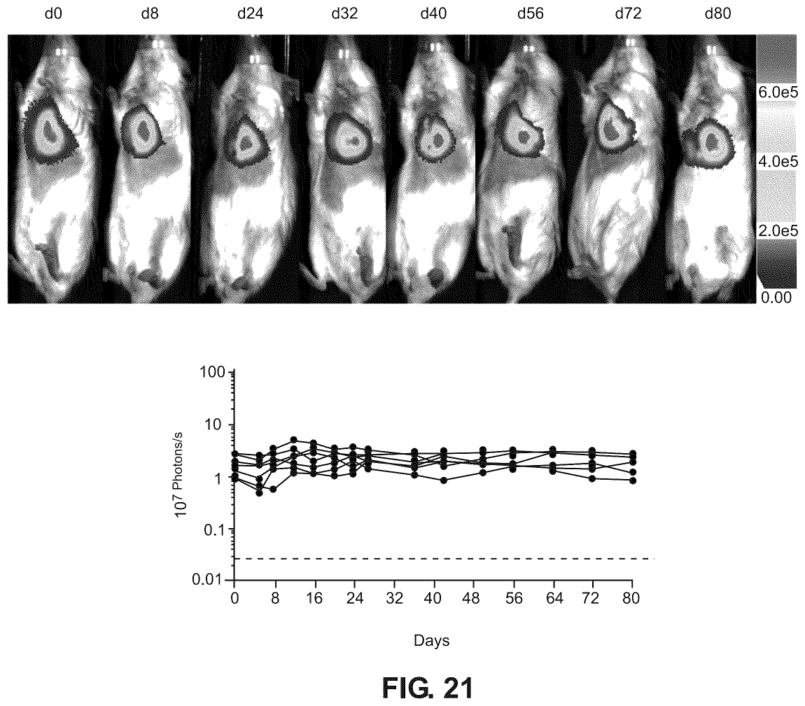

In addition, the cells can be tested to confirm that the HLA II complex is not expressed on the cell surface. Again, this assay is done as is known in the art (See FIG. 21 of PCT/US18/13688, for examplePCT/US18/13688) and generally is done using either Western Blots or FACS analysis based on commercial antibodies that bind to human HLA Class II HLA-DR, DP and most DQ antigens as outlined below.

A particularly useful embodiment uses CRISPR technology to disrupt the CIITA gene. CRISPRs were designed to target the coding sequence of the Ciita gene in mouse or the CIITA gene in human, an essential transcription factor for all MHC II molecules. After gene editing, the transfected iPSC cultures were dissociated into single cells. They were expanded to full-size colonies and tested for successful CRISPR editing by screening for the presence of an aberrant sequence from the CRISPR cleavage site. Clones with deletions did not express CIITA as determined by PCR and did not express MHC II/HLA-II as determined by FACS analysis.

3. Reduction of Macrophage Phagocytosis and/or NK Cell Killing

In addition to the reduction of HLA I and II (or MHC I and II), generally using B2M and CIITA knock-outs, the HIP cells of the invention have a reduced susceptibility to macrophage phagocytosis and NK cell killing. The resulting HIP cells “escape” the immune macrophage and innate pathways due to one or more CD47 transgenes.

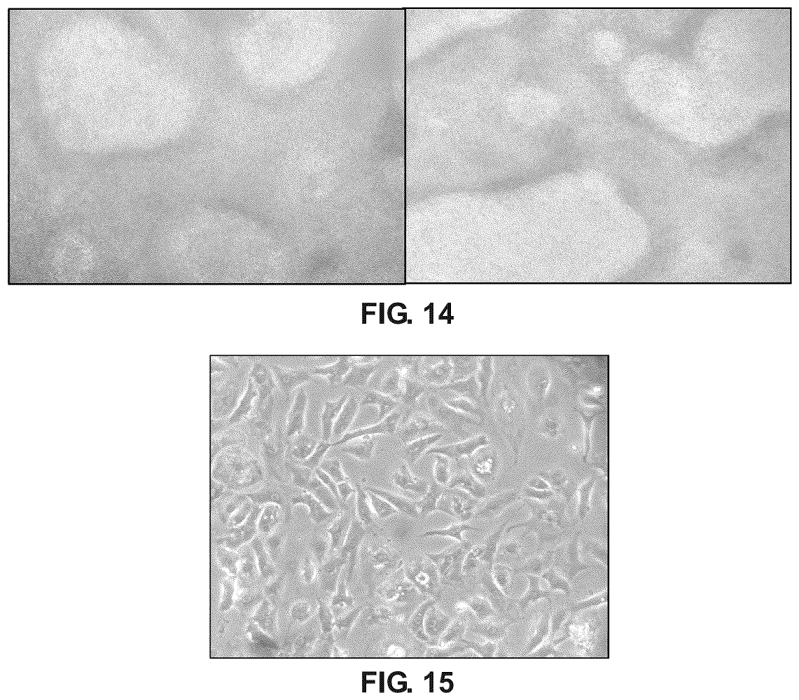

The ability of HIP cells and cells derived from the HIP cells to evade or escape NK cell killing and/or macrophage phagocytosis is shown in FIGS. 14A-14C and 34A-34C of PCT/US18/13688, the contents, in particular, the figures, figure descriptions, and examples are herein incorporated by reference. For example, FIGS. 14B-14C show that mouse HIP cells (e.g., B2m−/−Ciita−/−CD47 transgenic mouse iPSCs) failed to induce CD107a expression by NK cells, and thus did not elicit an NK cell response. In addition, it was shown that such mouse HIP cells did not induce activation of NK cells or release of IFNγ. When NK cells were incubated with differentiated cells (such as endothelial cells, smooth muscle cells, and cardiomyocytes) derived from HIP cells, NK cell responses were not induced (see, e.g., FIGS. 34A-34C of PCT/US18/13688).

### Increased CD47 Expression

In some embodiments, reduced macrophage phagocytosis and NK cell killing susceptibility results from increased CD47 on the HIP cell surface. This is done in several ways as will be appreciated by those in the art using “knock in” or transgenic technologies. In some cases, increased CD47 expression results from one or more CD47 transgene.

Accordingly, in some embodiments, one or more copies of a CD47 gene is added to the HIP cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The HIP cell lines were generated from B2M−/−CIITA−/−iPSCs. Cells containing lentivirus vectors expressing CD47 were selected using a blasticidin marker. The CD47 gene sequence was synthesized and the DNA was cloned into the plasmid Lentivirus pLenti6/V5 with a blasticidin resistance (Thermo Fisher Scientific, Waltham, MA)

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as those described in the Examples, such as Western blots, ELISA assays or FACS assays using anti-CD47 antibodies. In general, “sufficiency” in this context means an increase in the expression of CD47 on the HIP cell surface that silences NK cell killing and/or macrophage phagocytosis. The natural expression levels on cells is too low to protect them from NK cell lysis once their MHC I is removed.

### 4. Suicide Genes

In some embodiments, the invention provides hypoimmunogenic pluripotent cells (HIP cells) that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20 (10): 1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al. N. Engl. J. Med 365:18 (2011); Tey et al., Biol. Blood Marrow Transplant. 13:913-924 (2007), each of which are incorporated by reference herein in their entirety.)

### 5. Assays for HIP Cell Phenotypes and Retention of Pluripotency

Once the HIP cells have been generated, they may be assayed for their hypo-immunogenicity and/or retention of pluripotency as is generally described herein and in the examples.

For example, hypo-immunogenicity are assayed using a number of techniques as exemplified in FIG. 13 and FIG. 15 of PCT/US18/13688. These techniques include transplantation into allogeneic hosts and monitoring for HIP cell growth (e.g. teratomas) that escape the host immune system. HIP derivatives are transduced to express luciferase and can then followed using bioluminescence imaging. Similarly, the T cell and/or B cell response of the host animal to the HIP cells are tested to confirm that the HIP cells do not cause an immune reaction in the host animal. T cell function is assessed by Elispot, ELISA, FACS, PCR, or mass cytometry (CYTOF). B cell response or antibody response is assessed using FACS or luminex. Additionally or alternatively, the cells may be assayed for their ability to avoid innate immune responses, e.g. NK cell killing, as is generally shown in FIGS. 14A-14C of PCT/US18/13688. NK cell lytolytic activity is assessed in vitro or in vivo (as shown in FIG. 15).

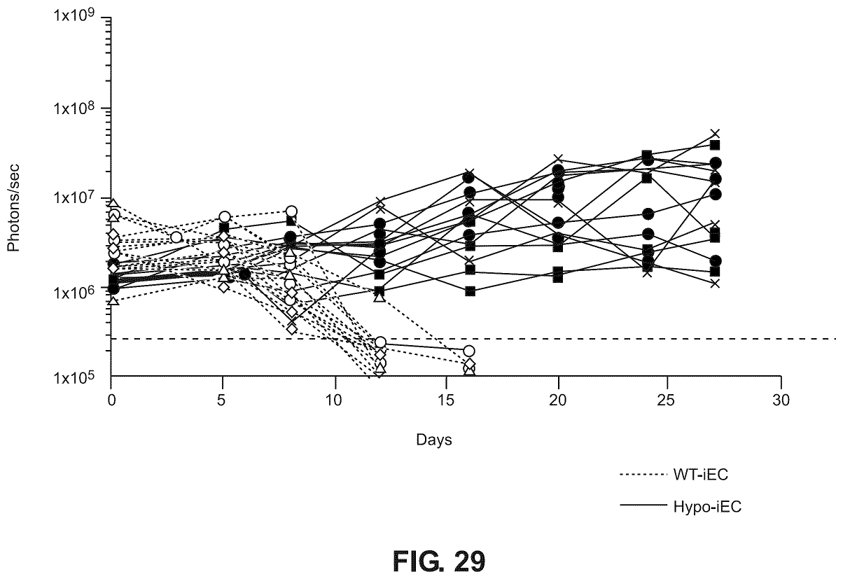

Similarly, the retention of pluripotency is tested in a number of ways. In one embodiment, pluripotency is assayed by the expression of certain pluripotency-specific factors as generally described herein and shown in FIG. 29 of PCT/US18/13688. Additionally or alternatively, the HIP cells are differentiated into one or more cell types as an indication of pluripotency.

### D. Preferred Embodiments of the HIP Cells

Provided herein are hypo-immunogenic pluripotent stem cells (“HIP cells”) that exhibit pluripotency but do not result in a host immune response when transplanted into an allogeneic host such as a human patient, either as the HIP cells or as the differentiated products of the HIP cells.

In one embodiment, human pluripotent stem cells (hiPSCs) are rendered hypo-immunogenic by a) the disruption of the B2M gene at each allele (e.g. B2M−/−), b) the disruption of the CIITA gene at each allele (e.g. CIITA−/−), and c) by the overexpression of the CD47 gene (CD47+, e.g. through introducing one or more additional copies of the CD47 gene or activating the genomic gene). This renders the hiPSC population B2M−/−CIITA−/−CD47tg. In a preferred embodiment, the cells are non-immunogenic. In another embodiment, the HIP cells are rendered non-immunogenic B2M−/−CIITA−/−CD47tg as described above but are further modified by including an inducible suicide gene that is induced to kill the cells in vivo when required.

### E. Maintenance of HIP Cells

Once generated, the HIP cells can be maintained an undifferentiated state as is known for maintaining iPSCs. For example, HIP cells are cultured on Matrigel using culture media that prevents differentiation and maintains pluripotency.

In some embodiments, the HIP cells are cryopreserved. The cells can be cryopreserved prior to differentiation into different cell types. In other words, prior to differentiation the HIP cells described herein are thawed and cultured before being subject to a differentiation method. In other embodiments, the HIP cells are not cryopreserved before differentiation. In some embodiments, the differentiated HIP cells are cryopreserved prior to administration to a patient. In other embodiments, the differentiated HIP cells are not cryopreserved before administration to a patient.

### F. Differentiation of HIP Cells

The invention provides HIP cells that are differentiated into different cell types for subsequent transplantation into subjects. As will be appreciated by those in the art, the methods for differentiation depend on the desired cell type using known techniques. The cells are differentiated in suspension and then put into a gel matrix form, such as matrigel, gelatin, or fibrin/thrombin forms to facilitate cell survival. Differentiation is assayed as is known in the art, generally by evaluating the presence of cell-specific markers.

In some embodiments, the HIP cells are differentiated into hepatocytes to address loss of the hepatocyte functioning or cirrhosis of the liver. There are a number of techniques that can be used to differentiate HIP cells into hepatocytes; see for example Pettinato et al., doi: 10.1038/spre32888, Snykers et al., Methods Mol Biol 698:305-314 (2011), Si-Tayeb et al. Hepatology 51:297-305 (2010) and Asgari et al., Stem Cell Rev (: 493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the HIP cells are differentiated into beta-like cells or islet organoids for transplantation to address type I diabetes mellitus (TIDM). Cell systems are a promising way to address TIDM, see. e.g., Ellis et al., doi/10.1038/nrgastro.2017.93, incorporated herein by reference. Additionally, Pagliuca et al. reports on the successful differentiation of β-cells from hiPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host: (doi: 10.1038/nm.4030, hereby incorporated by reference in its entirety and in particular for the methods and reagents outlined there for the large-scale production of functional human β cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi: 10.1016/j.cels.2016.09.002, hereby incorporated by reference in its entirety, and specifically for the biomarkers outlined there.

Once the dHIP beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

In some embodiments, the HIP cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi: 10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi: 10.1016/j.stemcr.2013.12.007, hereby incorporated by reference in its entirety and specifically for the markers outlined in the first paragraph of the results section.

In some embodiments, the HIP cells are differentiated into cardiomyocytes to address cardiovascular diseases. Techniques are known in the art for the differentiation of hiPSCs to cardiomyoctes and discussed in the Examples. Differentiation can be assayed as is known in the art, generally by evaluating the presence of cardiomyocyte associated or specific markers or by measuring functionally; see for example Loh et al., doi: 10.1016/j.cell.2016.06.001, hereby incorporated by reference in its entirety and specifically for the methods of differentiating stem cells including cardiomyocytes.

In some embodiments, the HIP cells are differentiated into endothelial colony forming cells (ECFCs) to form new blood vessels to address peripheral arterial disease. Techniques to differentiate endothelial cells are known. See. e.g., Prasain et al., doi: 10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the HIP cells are differentiated into thyroid progenitor cells and thyroid follicular organoids that can secrete thyroid hormones to address autoimmune thyroiditis. Techniques to differentiate thyroid cells are known the art. See. e.g. Kurmann et al., doi: 10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### VIII. HYPOIMMUNE CARDIAC CELLS DERIVED FROM HIP CELLS

In some embodiments, cardiac cells are derived from the HIP cells described herein. For instance, human cardiac cells can be produced by differentiating human HIP cells. Similarly, murine cardiac cells can be produced by differentiating murine HIP cells. Such cardiac cells are hypoimmune cardiac cells.

Useful method for differentiating induced or embryonic pluripotent stem cells into cardiac cells are described, for example, in US20170152485, US20170058263, US20170002325, US20160362661, US20160068814, U.S. Pat. Nos. 9,062,289, 7,897,389, and 7,452,718.

Additional methods for producing cardiac cells from induced or embryonic pluripotent stem cells are described in, for example, Xu et al, Stem Cells and Development, 2006, 15 (5): 631-9, Burridge et al., Cell Stem Cell, 2012, 10:16-28, and Chen et al., Stem Cell Res, 2015, 15 (2): 365-375.

In various embodiments, HIP cells (e.g., mouse HIP cells and human HIP cells) can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, SO3031 (KY01-I), SO2031 (KY02-I), and SO3042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP-4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells of the present invention can be cultured on a surface, such as a synthetic surface to support and/or promote differentiation of HIP cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra (ethylene glycol) diacrylate, glycerol dimethacrylate, 1,4-butanediol dimethacrylate,

[symbol]

[symbol]

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

Engineered cardiac cells of the present invention include, but are not limited to, cardiomyocytes, nodal cardiomyocytes, conducting cardiomyocytes, working cardiomyocytes, cardiomyocyte precursors, cardiomyocyte progenitor cell, cardiac stem cell, and cardiac muscle cells. In some embodiments, the cardiomyocyte precursor refers to cell that is capable (without dedifferentiation or reprogramming) of giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 3 or 5 markers) from the following list: cardiac troponin I (cTnI), cardiac troponin T (cTnT), sarcomeric myosin heavy chain (MHC), GATA-4, Nkx2.5, N-cadherin, β1-adrenoceptor (B1-AR), ANF, the MEF-2 family of transcription factors, creatine kinase MB (CK-MB), myoglobin, or atrial natriuretic factor (ANF). In some embodiments, the engineered cardiac cells demonstrate spontaneous periodic contractile activity. In some cases, when that cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are hypoimmune cardiac cells.

The efficacy of cardiac cells prepared as described herein can be assessed in animal models for cardiac cryoinjury, which causes 55% of the left ventricular wall tissue to become scar tissue without treatment (Li et al., Ann. Thorac. Surg. 62:654, 1996; Sakai et al., Ann. Thorac. Surg. 8:2074, 1999, Sakai et al., J. Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the engineered cardiac cells (e.g., hypoimmune cardiac cells) are administered to a patient, e.g., a human patient in need thereof. The cardiac cells can be administered to a patient suffering from pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some instances, the patient has had a myocardial infarction. In particular instances, the patient is undergoing coronary artery bypass surgery.

The engineered cardiac cells can be transplanted into the patient using well known surgical techniques for grafting tissue and/or isolated cells into a heart. In some embodiments, the cells are introduced into the patient's heart tissue by injection (e.g., intramyocardial injection, intracoronary injection, trans-endocardial injection, trans-epicardial injection, percutaneous injection), infusion, and implantation.

Administration (delivery) of the engineered cardiac cell include, but are not limited to, subcutaneous or parenteral including intravenous, intraarterial (e.g. intracoronary), intramuscular, intraperitoneal, intramyocardial, trans-endocardial, trans-epicardial, intranasal administration as well as intrathecal, and infusion techniques.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP): antineoplastic agents, steroids, and the like.

The effects of therapy according to the methods of the invention can be monitored in a variety of ways. For instance, an electrocardiogram (ECG) or holter monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holter monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

As will be appreciated by those in the art, the differentiated HIP derivatives are transplanted using techniques known in the art that depends on both the cell type and the ultimate use of these cells. In general, the differentiated HIP cells of the invention are transplanted either intravenously or by injection at particular locations in the patient. When transplanted at particular locations, the cells may be suspended in a gel matrix to prevent dispersion while they take hold.

### IX. ENDOTHELIAL CELLS DERIVED FROM HIP CELLS

In some embodiments, endothelial cells are derived from the HIP cells described herein. For instance, human endothelial cells can be produced by differentiating human HIP cells. Similarly, murine endothelial cells can be produced by differentiating murine HIP cells. Such endothelial cells are hypoimmune endothelial cells.

Useful method for differentiating induced or embryonic pluripotent stem cells into endothelial cells are described, for example, in US2004/0009589, WO2011/090684, and WO2012/006440.

In various embodiments, HIP cells (e.g., mouse HIP cells and human HIP cells) can be cultured in culture medium comprising a GSK3 inhibitor, an ALK inhibitor, a BMP pathway inhibitor, a ROCK inhibitor, a WNT signaling activator, a WNT signaling inhibitor, a WNT agonist, a WNT antagonist, a Src inhibitor, a EGFR inhibitor, a PCK activator, a cytokine, a growth factor, an endothelial cell differentiation compound, an endothelial cell promoting compound, and the like.

The WNT signaling activator (e.g., a GSK3 inhibitor) includes, but is not limited to, CHIR-99021. The PCK activator includes, but is not limited to, PMA. The WNT signaling inhibitor includes, but is not limited to, a compound selected from KY02111, SO3031 (KY01-I), SO2031 (KY02-1), and SO3042 (KY03-I), and XAV939. The Src inhibitor includes, but is not limited to, A419259. The EGFR inhibitor includes, but is not limited to, AG1478.

Non-limiting examples of an agent for generating an endothelial cell from an iPSC include activin A, BMP-4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells of the present invention can be cultured on a surface, such as a synthetic surface to support and/or promote differentiation of HIP cells into hypoimmune endothelial cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate,

utq-jym-qjsj lq-htq irfhw-qfyjlin-jym qjsj lq-htq inr jymfhw-qfyjlyjymf-jym-qjsj
lq-htq inr jymfhw-qfyj161; 2mj} fsjirtquwtut}-qfyj irfhw-qfyjlsjtujsy-qlq-htq
irfhw-qfyjlywrr jym-qtqwtufsj gjsitfyj irfhw-qfyjlywrr jym-qtuwtufsj jymt}-qfyj-6
JT4TM.r jym-qlywh-hq ': 37363571; hijhfsj2irr jymfstqirfhw-qfyjlsjtujsy-qlq-htq
jymt}-qfyj irfhw-qfyjlfsi ywr jym-qtquwtufsj ywfhw-qfyj 3 Fcrylate x-symjxrrji fx
pst|s rs ymj fwytwtgyfnsji kwtr f htr r jwhrfq{jsitwlxzhm fx Utq-xhnjshjx1Nsh31
Xrilr f FqiwhmlNsh3lfsi Xfwytr jwlNsh3

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacry late, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

Engineered endothelial cells of the invention can express one or more endothelial cell markers. Non-limiting examples of such markers include VE-cadherin (CD144), ACE (angiotensin-converting enzyme) (CD143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPASI (Endothelial PAS domain protein 1), Factor VIII related antigen, FLI-1, Flk-1 (KDR, VEGFR-2), FLT-1 (VEGFR-1), GATA2, GBP-1 (guany late-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEMI (Tumor endothelial marker 1), TEM5 (Tumor endothelial marker 5), TEM7 (Tumor endothelial marker 7), Thrombomodulin (TM, CD141), VCAM-1 (vascular cell adhesion molecule-1) (CD106), VEGF (Vascular endothelial growth factor), vWF (von Willebrand factor), ZO-1, endothelial cell-selective adhesion molecule (ESAM), CD102, CD93, CD184, CD304, and DLL4.

Endothelial cells include, but are not limited to, endothelial progenitor cells, capillary endothelial cells, arterial endothelial cells, venous endothelial cells, lymphatic vascular endothelial cells, other vascular endothelial cells, aortic endothelial cells, endothelial cells of the blood-brain barrier, cardiac endothelial cells, renal endothelial cells, and liver endothelial cells. Types and characteristics of different endothelial cells are described in Atkins et al., Arteriosclerosis, Thrombosis, and Vascular Biology, 2011, 31:1476-1484 and in U.S. Pat. No. 5,980,088. In some embodiments, the isolated, engineered endothelial cell of the invention is selected from the group consisting of a vascular endothelial cell, brain endothelial cell, renal endothelial cell, and aortic endothelial cell. In a preferred embodiment, the endothelial cell is a capillary endothelial cell.

In some embodiments, the engineered endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In certain embodiments, the engineered endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the engineered endothelial cells are genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, the engineered endothelial cells are administered to a patient, e.g., a human patient in need thereof. The endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In certain embodiments, the patient has suffered from or is suffering from a transient ischemic attack or stroke, which in some cases, may be due to cerebrovascular disease. In some embodiments, the engineered endothelial cells are administered to treat tissue ischemia e.g., as occurs in atherosclerosis, myocardial infarction, and limb ischemia and to repair of injured blood vessels. In some instances, the cells are used in bioengineering of grafts.

For instance, the engineered endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the endothelial cells can be further modified to deliver agents to target and treat tumors.

In specific embodiments, provided herein is a method of repair or replacement for tissue in need of vascular cells or vascularization. This method involves administering to a human patient in need of such treatment, a composition containing the isolated endothelial cells to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In certain embodiments, the engineered endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic.

The engineered endothelial cells can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the engineered endothelial cell include, but are not limited to, subcutaneous or parenteral including intravenous, intraarterial (e.g. intracoronary), intramuscular, intraperitoneal, intramyocardial, trans-endocardial, trans-epicardial, intranasal administration as well as intrathecal, and infusion techniques.

As will be appreciated by those in the art, the differentiated HIP derivatives are transplanted using techniques known in the art that depends on both the cell type and the ultimate use of these cells. In general, the differentiated HIP cells of the invention are transplanted either intravenously or by injection at particular locations in the patient. When transplanted at particular locations, the cells may be suspended in a gel matrix to prevent dispersion while they take hold.

### X. HYPOIMMUNE DOPAMINERGIC NEURONS DERIVED FROM HIP CELLS

In some embodiments, dopaminergic (DA) neurons are derived from the HIP cells described herein. For instance, human DA neurons can be produced by differentiating human HIP cells. Similarly, murine DA neurons can be produced by differentiating murine HIP cells. Such DA neurons are hypoimmune DA neurons.

Useful methods for differentiating pluripotent stem cells into DA neurons are described in, for example, U.S. Pat. Nos. 9,968,637, and 7,674,620, the disclosures in their entirety, including the specifications are herein incorporated by reference. Additional methods for producing DA cells from human pluripotent stem cells can be found in, for example, Kim, J.-H., et al., Nature, 2002, 418,50-56; Björklund, L. M., et al., PNAS, 2002, 99 (4), 2344-2349; Grow, D. A., et al., Stem Cells Transl Med. 2016, 5 (9): 1133-44, and Cho, M. S., et al., PNAS, 2008, 105:3392-3397.

The term “dopaminergic neurons” refers to neuronal cells which express tyrosine hydroxy lase (TH), the rate-limiting enzyme for dopamine synthesis. Preferably dopaminergic neurons secrete the neurotransmitter dopamine, and have little or no expression of dopamine-β-hydroxylase. A dopaminergic neuron can express one or more of the following: neuron-specific enolase (NSE), 1-aromatic amino acid decarboxylase, vesicular monoamine transporter 2, dopamine transporter, Nurr-1, and dopamine-2 receptor (D2 receptor). A dopaminergic neuron includes a neuronal stem cell, neuronal progenitor cell, immature dopaminergic neuron, and mature dopaminergic neuron.

The term “neural stem cells” refers to a subset of pluripotent cells which have partially differentiated along a neural cell pathway and express some neural markers including, for example, nestin. Neural stem cells may differentiate into neurons or glial cells (e.g., astrocytes and oligodendrocytes). The term “neural progenitor cells” refers to cultured cells which express FOXA2 and low levels of β-tubulin. but not tyrosine hydroxylase (i.e., having a FOXA2+/β-tubulinLO/TH− phenotype). Such neural progenitor cells have the capacity to differentiate into a variety of neuronal subtypes: particularly a variety of dopaminergic neuronal subtypes, upon culturing the appropriate factors, such as those described herein.

HIP cells and DA neurons derived from HIP cells can be cultured in a growth media. Illustrative growth media include, but are not limited to, human embryonic stem cell medium (hESC medium), Dulbecco's Modified Eagle Medium mammalian cell culture medium (DMEM), Ham's F12 medium, Neurobasal™ (ThermoFisher), Knockout Serum Replacer (KOSR), Minimum Essential Medium Eagle-alpha modification (Alpha MEM), Knockout DMEM (KO-DMEM), N-2 (ThermoFisher), MS-5 stromal cell culture medium, and the like.

Useful additives that promote differentiation, growth, expansion, maintenance, and/or maturation of DA neurons include, but are not limited to, Wnt1, fibroblast growth factor 2 (FGF2), FGF8, FGF8a, Sonic Hedgehog (SHH), brain derived neurotrophic factor (BDNF), transforming growth factor α (TGF-α), TGF-β3, interleukin 1 beta (IL1β), glial cell line-derived neurotrophic factor (GDNF), a GSK-3 inhibitor (e.g., CHIR-99021), a TGF-β inhibitor (e.g., SB-431542), B-27 supplement, dorsomorphin, purmorphamine, noggin, retinoic acid, cAMP, ascorbic acid, GlutaMax™, neurturin, Knockout Serum Replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, TGFβ pathway, and the like. Differentiation protocols and detailed descriptions thereof are provided in, e.g., U.S. Pat. Nos. 9,968,637, and 7,674,620, Kim, J.-H., et al., Nature, 2002, 418,50-56; Björklund, L. M., et al., PNAS, 2002, 99 (4), 2344-2349; Grow, D. A., et al., Stem Cells Transl Med. 2016, 5 (9): 1133-44, and Cho, M. S., et al., PNAS, 2008, 105:3392-3397, the disclosures in their entirety including the detailed description of the invention, example, methods, online methods, and results are herein incorporated by reference.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like.

Exemplary markers for DA neurons include TH, β-tubulin, paired box protein (Pax6), insulin gene enhancer protein (ISL1), nestin, diaminobenzidine (DAB), G protein-activated inward rectifier potassium channel 2 (GIRK2), microtubule-associated protein 2 (MAP-2), nuclear receptor related 1 protein (NURRI), dopamine transporter (DAT), forkhead box protein A2 (FOXA2), FOX3, doublecortin, and LIM homeobox transcription factor 1-beta (LMX1B), and the like.

DA neurons can also be assessed according to cell electrophysiological makers. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the DA neurons derived from HIP cells are administered to a patient, e.g., human patient to treat a neurodegenerative disease or condition. In some cases, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

The differentiated DA neurons can be transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease (PD). The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, specifically incorporated herein by reference. In some embodiments, the differentiated DA neurons are supplied in the form of a pharmaceutical composition. The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration.

As will be appreciated by those in the art, the differentiated HIP derivatives are transplanted or grafted using techniques known in the art that depends on both the cell type and the ultimate use of these cells. In general, the differentiated HIP cells of the invention are transplanted or injected at particular locations in the patient. When transplanted at particular locations, the cells may be suspended in a gel matrix to prevent dispersion while they take hold.

### XI. HYPOIMMUNE PANCREATIC ISLET CELLS DERIVED FROM HIP CELLS

In some embodiments, pancreatic islet cells (also referred to as pancreatic beta cells) are derived from the HIP cells described herein. For instance, human pancreatic islet cells can be produced by differentiating human HIP cells. Similarly, murine pancreatic islet cells can be produced by differentiating murine HIP cells. Such pancreatic islet cells are hypoimmune pancreatic islet cells.

In some embodiments, pancreatic islet cells are derived from the HIP cells described herein. Useful method for differentiating pluripotent stem cells into pancreatic islet cells are described, for example, in U.S. Pat. Nos. 9,683,215, 9,157,062, and 8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdx1, glucose transporter 2 (Glut2), HNF6, VEGF, glucokinase (GCK), prohormone convertase (PC1/3), Cdcp1, NeuroD, Ngn3, Nkx2.2, Nkx6.1, Nkx6.2, Pax4, Pax6, Ptfla, Isl1, Sox9, Sox 17, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 μm to about 25 μm.

In some embodiments, the method of differentiating HIP cells into hypoimmune pancreatic islet cells comprising culturing the HIP stem cells in culture medium comprising FGF10. In some cases, the culture medium comprising one or more differentiation factors selected from the group consisting of keratinocyte growth factor (KGF), epidermal growth factor (EGF): transforming growth factor-α (TGFα), transforming growth factor-β (TGFβ), hepatocyte growth factor (HGF), Wnt3a, Activin A, Nodal, KAAD-CYC, (basic fibroblast growth factor (bFGF), nicotinamide, indolatam V, an HDAC inhibitor, IDE1, and IDE2. In some embodiments, HIP cells are differentiated to pancreatic islet cells by culturing the cells in culture medium comprising one or more of the following: insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-β (TGFß) superfamily, bone morphogenic protein-2 (BMP2), bone morphogenic protein-7 (BMP7), a GSK3B inhibitor, an ALK inhibitor, a BMP type 1 receptor inhibitor, retinoic acid, and any combination thereof.

In some embodiments, a population of hypoimmune pluripotent stem cells (HIP cells) can be contacted or exposed to one or more of the compounds of Formula (I), e.g. IDE1 or IDE2 as described herein alone, and in other embodiments, a population of pluripotent stem cells can be contacted with at least one additional agent, either concurrent with (e.g. in combination with), subsequent to or prior to the contact of the pluripotent cell with a compound of Formula (I) as disclosed in U.S. Pat. No. 8,927,280.

In some embodiments, the additional compound for use in combination with compounds of Formula (I) as disclosed in U.S. Pat. No. 8,927,280 can include, but is not limited to agents of transforming growth factor-β (TGFβ) family member (e.g., Nodal or Activin A), fibroblast growth factor (FGF) family member (e.g., FGF10), Wnt growth factor family member (e.g., Wnt3a), bone morphogenic proteins (BMPs) and/or members of the AKT/PI3K pathway. The definition and details of the TGF-beta3/BMP pathway are disclosed in the art e.g., Kawabata M. and Miyazono K., J. Biochem. (Tokyo), 125, 9-16 (1999): Wrana J. L., Miner. Electrolyte Metab., 24, 120-130 (1998); and Markowitz S. D., and Roberts A. B., Cytokine Growth Factor Rev., 7, 93-102 (1996). In some embodiments, a pluripotent stem cell can be exposed to a compound of Formula (I), e.g. IDE1 and/or IDE2 in combination with at least one additional compounds or factors including, but not limited to cyclopamine, TGF family members (TGF-alpha, Activin A, Activin B, TGF-β-1, TGF-beta-3), exendin 4, nicotinamide, n-butyrate, DMSO, all-trans retinoic acid, GLP-I, bone morphogenic proteins (BMP-2, BMP-5, BMP-6, BMP-7), insulin-like growth factors (IGF-I, IGF-II), fibroblast growth factor (FGF7, FGF10, bFGF, FGF4), other growth factors (EGF, beta cellulin, growth hormone, HGF), other hormones (prolactin, cholecytokinin, gastrin I, placental lactogen), TGF-β family antagonists (Noggin, follistatin, chordin), IBMX, wortmannin, dexamethazone, Reg, INGAP, CAMP or cAMP activators (forskolin), and/or extracellular matrix components (laminin, fibronectin).

In some embodiments, the HIP cell is contacted with at least one histone deacetylase (HDAC) inhibitor (e.g., a class I/II HDAC inhibitor) to differentiate the cell a pancreatic islet cell. Histone deacetylase (HDAC) are a class of enzymes that remove acetyl groups from an e-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

A number of structural classes of negative regulators of HDACs (e.g., HDAC inhibitors) have been developed, for example, small molecular weight carboxylates (e.g., less than about 250 amu), hydroxamic acids, benzamides, epoxyketones, cyclic peptides, and hybrid molecules. (See, for example, Drummond D C, Noble C O, Kirpotin D B, Guo Z. Scott G K, et al. (2005) Clinical development of histone deacety lase inhibitors as anticancer agents. Annu Rev Pharmacol Toxicol 45:495-528, (including specific examples therein) which is hereby incorporated by reference in its entirety). Non-limiting examples of negative regulators of type I/II HDACs include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de] isoquinolin-2-yl)-N-hydroxy butanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8. Apicidin, Sodium Butyrate, pivaloyloxymethyl butyrate (Pivanex, AN-9). Trapoxin B. Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-CI-UCHA (i.e., 6-(3-chlorophenylureido) caproic hydroxamic acid), AOE (2-amino-8-oxo-9,10-epoxydecanoic acid), CHAP31, CHAP 50, IDE1 and IDE2. Other inhibitors include, for example, dominant negative forms of the HDACs (e.g., catalytically inactive forms) siRNA inhibitors of the HDACs, and antibodies that specifically bind to the HDACs. Inhibitors are commercially available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. In some embodiments, IDE1 or IDE2 is a preferred histone deacetylase inhibitor.

Differentiation of the HIP cells can be achieved by contacting, e.g., overlaying, a monolayer of HIP cells with a component or components of the extracellular matrix (ECM). In some embodiments, the layer of HIP cells is contacted with an extracellular matrix component which is one or more of: laminin, e.g., laminin 1: collagen, e.g., collagen IV: entactin: heparin sulfate proteoglycan: nidogen. The extracellular matrix component can be a basement membrane derived substance, e.g., a basement membrane laid down by a cell, e.g., a tumor cell, e.g., an Engelbreth-Holm-Swarm (EHS) tumor cell. In some embodiments, the extracellular matrix component is Matrigel™ which is commercially available from Becton-Dickenson. The extracellular component can further include: one or more growth factor(s), one or more matrix metalloproteinase(s) (MMP), e.g., MMP-2, MMP-3, and combinations thereof.

The HIP cells can be cultured in the presence of the extracellular matrix or component or components of the extracellular matrix for a period of at least 1, 2, 3, 5, 7, 10, 12, 14, 16, 18, 21, 25, 28, 30, 35, 40, 42, 48, 50 or more days.

In some embodiments, the HIP-derived pancreatic islet cells can be administered to a patient, e.g., a human patient in need thereof. In some instances, the patient has a disease, disorder, or condition that can be treated using such cells. In other words, administration of the HIP-derived pancreatic islet cells can reduce or alleviate at least one adverse effect or symptom associated with insulin metabolism as is well-known in the art. In some embodiments, the patient has a disease characterized by insufficient insulin activity which can include diseases in which there is an abnormal utilization of glucose due to abnormal insulin function. Abnormal insulin function may include any abnormality or impairment in insulin production (e.g., expression and/or transport through cellular organelles, such as insulin deficiency resulting from, for example, loss of β cells): secretion (e.g., impairment of insulin secretory responses): the form of the insulin molecule itself (e.g., primary, secondary or tertiary structure): effects of insulin on target cells (e.g., insulin-resistance in bodily tissues including peripheral tissues); and responses of target cells to insulin.

Common methods of administering pancreatic islet cells to subjects, particularly human subjects, are described herein. For example, pancreatic islet cells can be administered to a subject by injection or implantation of the cells into target sites in the subjects. In addition, the cells can be inserted into a delivery device which facilitates introduction by injection or implantation of the cells in the subjects. Such delivery devices include tubes, e.g., catheters for injecting cells and fluids in to the body of a recipient subject. In a preferred embodiment, the tubes additionally have a needle, e.g., a syringe, through which the cells of the invention can be introduced into the subject at a desired location. The pancreatic islet cells can be inserted into such a delivery device, e.g., a syringe, in different forms. For example, the cells can be suspended in a solution or embedded in a support matrix when contained in such a delivery device.

As used herein the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that the cells and solution can be pass through a syringe. In some embodiments, the solution is stable under the conditions of manufacture and storage and preserved against the contaminating action of microorganisms such as bacteria and fungi through the use of, for example, parabens, chlorobutanol, phenol, ascorbic acid, thimerosal and the like. Such solutions can be prepared by incorporating the pancreatic islet cells described herein in a pharmaceutically acceptable carrier or diluent, followed by filtered sterilization.

Support matrices in which the pancreatic islet cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from mammal, and collagen matrices. Synthetic biodegradable matrices include synthetic polymers such as polyanhydrides, polyorthoesters, and polylactic acid. Other examples of synthetic polymers and methods of incorporating or embedding cells into the matrices are known in the art. See e.g., U.S. Pat. Nos. 4,298,002 and 5,308,701. The matrices provide support and/or protection for the fragile pancreatic cells in vivo.

As will be appreciated by those in the art, the differentiated HIP derivatives are transplanted or grafted using techniques known in the art that depends on both the cell type and the ultimate use of these cells. In general, the differentiated HIP cells of the invention are transplanted or injected at particular locations in the patient. When transplanted at particular locations, the cells may be suspended in a gel matrix to prevent dispersion while they take hold.

### XII. HYPOIMMUNE RETINAL PIGMENTED EPITHELIUM (RPE) CELLS DERIVED FROM HIP CELLS

In some embodiments, retinal pigmented epithelium (RPE) cells are derived from the HIP cells described herein. For instance, human RPE cells can be produced by differentiating human HIP cells. Similarly, murine RPE cells can be produced by differentiating murine HIP cells. Such RPE cells are hypoimmune RPE cells.

HIP cells described herein can be differentiated into retinal pigmented epithelium (RPE) cells including RPE progenitor cells, immature RPE cells, mature RPE cells, and functional RPE cells.

Useful methods for differentiating embryonic pluripotent stem cells into RPE cells are described in, for example, U.S. Pat. Nos. 9,458,428, and 9,850,463, the disclosures in their entirety, including the specifications are herein incorporated by reference. Additional methods for producing RPE cells from human embryonic or induced pluripotent stem cells can be found in, for example, Lamba et al., PNAS, 2006, 103 (34): 12769-12774: Mellough et al., Stem Cells, 2012, 30 (4): 673-686; Idelson et al., Cell Stem Cell, 2009, 5 (4): 396-408; Rowland et al., Journal of Cellular Physiology, 2012, 227 (2): 457-466, Buchholz et al., Stem Cells Trans Med, 2013, 2 (5): 384-393, and da Cruz et al., Nat Biotech, 2018, 36:328-337.

The term “RPE” cells refers to pigmented retinal epithelial cells having a genetic expression profile similar or substantially similar to that of native RPE cells. Such RPE cells derived from pluripotent stem cells may possess the polygonal, planar sheet morphology of native RPE cells when grown to confluence on a planar substrate.

HIP cells and RPE cells derived from HIP cells can be cultured in a growth media. Illustrative growth media include, but are not limited to, X-VIVO 10™ (Lonza Biosciences), X-VIVO 15™ (Lonza Biosciences), MTESR2™ (Stem Cell Technologies), NUTRISTEM™ (StemGent) and HESCGRO™ (Millipore). Lonza X-VIVO 10™ supplemented with 5-40% Xeno-Free Knockout Serum Replacement (XF-KOSR™. Invitrogen), MX-302 (Iscove's Modified Dulbecco's Medium (IMDM) with B-27 supplement), Essential 8™ medium, Dulbecco's Modified Eagle Medium mammalian cell culture medium (DMEM), Ham's F12 medium, Iscove's Modified Dulbecco's Medium (IMDM), Minimum Essential Medium Eagle (MEM), Roswell Park Memorial Institute Medium 1640 (RPMI-1640), MCDB medium, and the like.

Useful additives that promote differentiation, growth, expansion, maintenance, and/or maturation of RPE cells include, but are not limited to, an inhibitor of BMP signaling (e.g., LDN-193189, dorsomorphin, chordin, cerburus, and noggin), an inhibitor of WNT signaling (e.g., Dickkopf-related protein (DKK1), IWP-2, IWP-3, IWP-4, XAV939, decreted frizzled related protein (SFRP1 and SFRP2), and Wnt Inhibitory Factor 1 (WIF-1)), an inhibitor of FGF signaling (e.g., SU5402, AZD4547, and PD173074), insulin-like growth factor (IGF1), nicotinamide, benzoic acid, 3-aminobenzoic acid, 6-aminonicotinamide, an inhibitor of poly(ADP-ribose) polymerase (PARP) (e.g., 3-aminobenzamide, iniparib (BSI 201), olaparib (AZD-2281), and rucaparib (AG014699, PF-01367338), veliparib (ABT-888), CEP 9722, MK 4827, and BMN-673), a ROCK inhibitor (e.g., Y-27632, thiazovivin, GSK429286A, and Fasudil), basic fibroblast growth factor 1 (bFGF1), FGF1, FGF2, FGF3, FGF4, FGF5, FGF6, FGF7, FGF8, FGF9, FGF10, SUN13837, F2A4-K-NS, trichostatin A, activin A, activin AB, activin B. BMP-4, BMP-7, TGF-β1, vasoactive intestinal peptide (VIP). forskolin, rolipram, B-27 supplement, Knockout Serum Replacement, N2 supplement, taurine, progesterone, vitamin A, blebbistatin, modified forms thereof, mimics thereof, analogs thereof, and variants thereof. Differentiation protocols and detailed descriptions thereof are provided in, e.g., U.S. Pat. Nos. 9,458,428 and 9,850,463, and da Cruz et al., Nat Biotech, 2018, 36:328-337, the disclosures in their entirety including the detailed description of the invention, example, methods, online methods, and results are herein incorporated by reference.

In some embodiments, the hypoimmune RPE cells are differentiated, propagated, or immobilized on a cell culture substrate. Exemplary cell culture substrates commonly-used substrates such as Matrigel™ (Corning Life Sciences), mouse embryonic fibroblast feed cell layers, human embryonic fibroblasts, human fallopian tube epithelium, or human foreskin fibroblasts feeder layers. Xeno-free substrates can also be used such as, but not limited to, Synthemax™ (Corning Life Sciences), CELLstart™ (Invitrogen), GELstart™ (Invitrogen), and StemAdhere™ (Primorigen). Additional cell culture substrates may comprise one or more of the following including purified human vitronectin, recombinant human vitronectin, recombinant human fibronectin (e.g., RetroNectin®; Takara Bio), purified human laminin, recombinant laminin, recombinant laminin 511, recombinant laminin 521, poly-D-lysine, and the like.

In certain embodiments, the RPE cells are differentiated, propagated, or immobilized on a biocompatible substrate such as a synthetic substrate. Non-limiting substrates include polymeric substrates, polyester membranes, polyethylene terephthalate (PET) membranes, poly(DL-lactic-co-glycolic acid) (PLGA) membranes, expanded polytetrafluoroethylene (ePTFE) membranes, polycaprolactone membranes, electrospun artificial scaffolds produced from methylmethacrylate and poly(ethylene glycol), and the like. Exemplary substrates are described in, for example, U.S. Pat. No. 8,808,687, the disclosure in its entirety is herein incorporated by reference. Illustrative examples of suitable materials for the substrate include, but are not limited to, parylene polypropylene, polyimide, glass, nitinol, polyvinyl alcohol, polyvinyl pyrolidone, collagen, chemically-treated collagen, polyethersulfone (PES), poly(glycerol-sebacate) PGS, poly(styrene-isobutyl-styrene), polyurethane, ethyl vinyl acetate (EVA), polyetherether ketone (PEEK), Kynar (Polyvinylidene Fluoride: PVDF), Polytetrafluoroethylene (PTFE), Polymethylmethacrylate (PMMA), Pebax, acrylic, polyolefin, polydimethylsiloxane (PDMS) and other silicone elastomers, polypropylene, hydroxyapetite, titanium, gold, silver, platinum, other metals and alloys, ceramics, plastics and mixtures or combinations thereof. Additional suitable materials used to construct a substrate include, but are not limited to, poly-para-xylylenes (e.g., parylene, including but not limited to pary lene A. parylene AM, parylene C, ammonia treated parylene, parylene C treated with polydopamine), poly(lactic acid) (PLA), polyethylene-vinyl acetate, poly(lactic-co-glycolic acid) (PLGA), poly(D,L-lactide), poly(D,L-lactide-co-trimethylene carbonate), collagen, heparinized collagen, denatured collagen, modified collaged (e.g., silicone with gelatin), other cell growth matrices (such as SYNTHEMAX™), poly(caprolactone), poly(glycolic acid), and/or other polymer, copolymers, or block co-polymers, poly(caprolactone) containing cyclic arginine-glycine-asparagine, cyclic or linear arginine-glycine-aspartic acid, blends of polycaprolactone and polyethylene glycol (PCL-PEG), thermoplastic polyurethanes, silicone-modified polyether urethanes, poly(carbonate urethane), or polyimide. Exemplary thermoplastic polyurethanes are polymers or copolymers which may comprise aliphatic polyurethanes, aromatic polyurethanes, polyurethane hydrogel-forming materials, hydrophilic polyurethanes, or combinations thereof. Non-limiting examples include elasthane (poly(ether urethane)) such as Elasthane™ 80A, Lubrizol, Tecophilic™, Pellethane™, Carbothane™, Tecothane™, Tecoplast™, and Estane™ Silicone-modified polyether urethanes may include Carbosil™ 20 or Pursil™ 20 80A, and the like. Poly(carbonate urethane) may include Bionate™ 80A or similar polymers.

In some embodiments, the substrate is biodegradable. In other embodiments the substrate is non-biodegradable. In particular embodiments, the substrate comprises one or more biodegradable components and one or more non-biodegradable components.

To characterize and monitor RPE differentiation and assess the RPE phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassay's, immunocytochemistry assays, immunoblotting assays, and the like.

Exemplary markers for RPE cells include Paired box protein (Pax6), Rax homeobox protein (Rax), LIM/homeobox protein 2 (Lhx2), homeobox protein SIX3, tyrosinase enzyme (TYR), microphthalmia-associated transcription factor (MITF), cellular retinaldehyde-binding protein (CRALBP), trypsin-1 (cationic trypsinogen, TYRP1), trypsin-2 (anionic trypsinogen, TYRP2), premelanosome protein (PMEL17), silver locus protein homolog (SILV), ceh-10 homeodomain containing homolog (Chx10), bestrophin-1 (BEST), and retinal pigment epithelium-specific 65 kDa protein (RPE65).

RPE cells can also be assessed according to cell physiological markers and morphological markers. Immunocytochemistry and electron microscopy can be used to determine morphology of the cells. RPE cells can be evaluated using functional assays known to those skilled in the art. For instance, pigment-epithelium-derived factor (PEDF) secretion profiling, phagocytosis of rod outer segments (ROS) assays, assays for trans retinol conversion to 11-cis retinol, assays for determining the polarized secretion of growth factors, and assays for detecting tight junctions that create an electrical barrier can be used to characterize the RPE cells derived from HIP cells.

In some embodiments of differentiation, the pluripotent stem cells undergo neural induction and express one or more retinal progenitor markers Pax6, Rax, Lhx2, Six3, or any other molecular, physiological, or morphological markers of neural induction. In other embodiments, the differentiating cells undergo RPE specification and/or form rosette structures. As the cells continue to differentiate, the rosette structures may flatten into a layer or a sheet of immature RPE cells. The layer of immature RPE cells may comprise planar cells with a polygonal and/or hexagonal shape.

In some embodiments, the hypoimmune RPE is implanted to a patient in need thereof. The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-realted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

The RPE cells can be immobilized on any of the substrates described herein to produce a RPE patch that can be transplanted into a patient in need thereof. The patch comprising one or more layers of RPE cells can be surgically administered or delivered to an ocular tissue. In some instances, patches are delivered to the neural retina or subretinal space. In certain embodiments, patches are delivered endoscopically, via catheter-based methods, intravascularly, intramuscularly, or by other means known in the art for a particular ocular tissue. Placement of patches can be determined using stereobiomicroscopy, fundus photography, spectral domain optical coherence tomography (SD-OCT), and other methods recognized by those in the art.

As will be appreciated by those in the art, the differentiated HIP derivatives are transplanted or grafted using techniques known in the art that depends on both the cell type and the ultimate use of these cells. In general, the differentiated HIP cells of the invention are transplanted or injected at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### XIII. EXAMPLES

### Example 1: Generation of Mouse Induced Pluripotent Stem Cells

The method described herein is adapted from Diecke et al., Sci Rep, 2015, 8081.

Murine tail tip fibroblasts of mice were dissociated and isolated with collagenase type IV (Life Technologies, Grand Island, NY, USA) and maintained with Dulbecco's modified Eagle medium (DMEM) containing 10% fetal bovine serum (FBS), L-glutamine, 4.5 g/L glucose, 100 U/mL penicillin, and 100 μg/mL streptomycin at 37° C., 20% O2, and 5% CO2 in a humidified incubator.

1×10⁶ murine fibroblasts were then reprogrammed using a novel codon optimized mini-intronic plasmid (co-MIP) (10-12 μm of DNA) expressing the four reprogramming factors Oct4, KLF4, Sox2 and c-Myc using the Neon Transfection system. After transfection, fibroblasts were plated on a murine embryonic fibroblasts (MEF) feeder layer and kept in fibroblast media with the addition of sodium butyrate (0.2 mM) and 50 μg/mL ascorbic acid.

When ESC-like colonies appeared, media was changed to murine iPSC media containing DMEM, 20% FBS, L-glutamine, non-essential amino acids (NEAA), β-mercaptoethanol, and 10 ng/ml leukemia inhibitory factor (LIF). After 2 passages, the murine iPSCs were transferred to 0.2% gelatin coated plates and further expanded. With every passage, the iPSCs were sorted for the murine pluripotency marker SSEA-1 using magnetic activated cell sorting (MACS).

The isolated mouse iPSCs can be used to generate mouse hypoimmunogenic iPSCs according to the method described above.

### Example 2: Generation of Human Induced Pluripotent Stem Cells

The Gibco™ Human Episomal iPSC Line (catalog number A18945, ThermoFisher) was derived from CD34+ cord blood using a three-plasmid, seven-factor (SOKMNLT: SOX2, OCT4 (POU5F1), KLF4, MYC, NANOG, LIN28, and SV40L T antigen) EBNA-based episomal system. This iPSC line is considered to be zero foot-print as there was no integration into the genome from the reprogramming event. It has been shown to be free of all reprogramming genes. Protocols for thawing, culturing, and passaging the human iPSCs are provided in the product manual.

Pluripotency of the human iPSCs can be determined by in vivo teratoma assays and in vitro pluripotent gene expression assays (e.g., PCR and arrays) or by fluorescence staining for pluripotent markers.

The Gibco™ Human Episomal iPSC Line has a normal karyotype and endogenous expression of pluripotent markers like OCT4, SOX2, and NANOG (as shown by RT-PCR) and OCT4, SSEA4, TRA-1-60 and TRA-1-81 (as shown by ICC). Whole genome expression and epigenetic profiling analyses demonstrated that this episomal hiPSC line is molecularly indistinguishable from human embryonic stem cell lines (Quintanilla et al., PloS One, 2014, 9 (1): e85419). In directed differentiation and teratoma analyses, these hiPSCs retained their differentiation potential for the ectodermal, endodermal, and mesodermal lineages (Burridge et al., PLoS One, 2011, 6 (4): e18293). In addition, vascular, hematopoietic, neural, and cardiac lineages were derived with robust efficiencies (Burridge et al., supra).

TABLE 1
Illustrative protocol for culturing human iPSCs (e.g., Cas9 iPSCs)

| Title | Human iPSC culture | |
|---|---|---|
| Introduction | Induced Pluripotent stem cells have the capacity to give rise to | |
| | differentiated progeny representative of all three germ layers (ectoderm, | |
| | endoderm, and mesoderm). The ability to expand pluripotent cells in | |
| | vitro and subject them to direct differentiation to produce specific cell | |
| | types is crucial to the development of cell-based therapies to replace or | |
| | restore tissue that has been damaged by disease or injury. | |
| Materials | 1. | Essential 8 Flex Media (Thermo Fisher Scientific, cat. no. |
| | | A2858501) |
| | 2. | Revita Cell Supplement (Thermo Fisher Scientific, cat. no. |
| | | A2644501) |
| | 3. | diluted Matrigel (Corning, cat. no. 356231), diluted in Knockout |
| | | DMEM (Thermo Fisher Scientific, cat. no. 10829) |
| | 4. | Versene (Thermo Fisher Scientific, cat. no. 15040066) |
| | 5. | 10 cm² cell culture plates (Corning, cat. no. 353003) |

| Protocol | | Notes |
|---|---|---|
| 1. | Thaw 1x vial in 1x 10 cm² dish, after approx. 4-5 | |
| | days, the cells will reach 60-70% confluency and | |
| | are ready for splitting | |
| 2. | Reconstitute Matrigel 1:40 in cold Knockout | |
| | DMEM and mix well. Place dishes in 37° C. | |
| | incubator for 30 minutes to use plates immediately | |
| | or seal with parafilm and store at 2-8° C. for up to | |
| | 7 days. | |
| 3. | Culture Cas9 human iPSC on diluted Matrigel | Incubate cells at |
| | (1:40 in KO DMEM) coated 10 cm² dishes in | 37° C./5% CO₂. |
| | Essential 8 Flex Media. | |
| 4. | Change media daily and passage the cells every 3- | Pipette gently. Do |
| | 4 days 1:6 using Versene for 9 min at 37° C. | not vortex!! |
| | | Centrifugation |
| | | 800 rpm, 4 min at |
| | | 4° C. |
| 5. | Revita Cell Supplement was added 1:100 in the | |
| | media after splitting for the first 24 h. | |

The isolated human iPSCs can be used to generate human hypoimmunogenic iPSCs (HIP cells) according to the method described above.

### Example 3: Hypoimmunogenic Pluripotent Cells were Less Susceptible to NK Cell Killing and Macrophage Phagocytosis

Examples were performed to evaluate the ability of hypoimmunogenic pluripotent cells (e.g., mouse b2m−/−ciita−/−CD47 tg iPSCs and human B2M−/−CIITA−/−CD47 tg iPSCs) and to evade the immune innate response pathways.

In particular, enzyme-linked immunospot (Elispot) assays were performed. NK cells were co-cultured with mouse HIP cells or human HIP cells (mouse B2m−/−Ciita−/−CD47 tg iPSCs or human B2M−/−CIITA−/−CD47 tg iPSCs) and IFNγ release was measured (e.g., innate IFNγ spot frequencies were measured using an Elispot plate reader). In some examples, CD47 was blocked by using an anti-CD47 antibody.

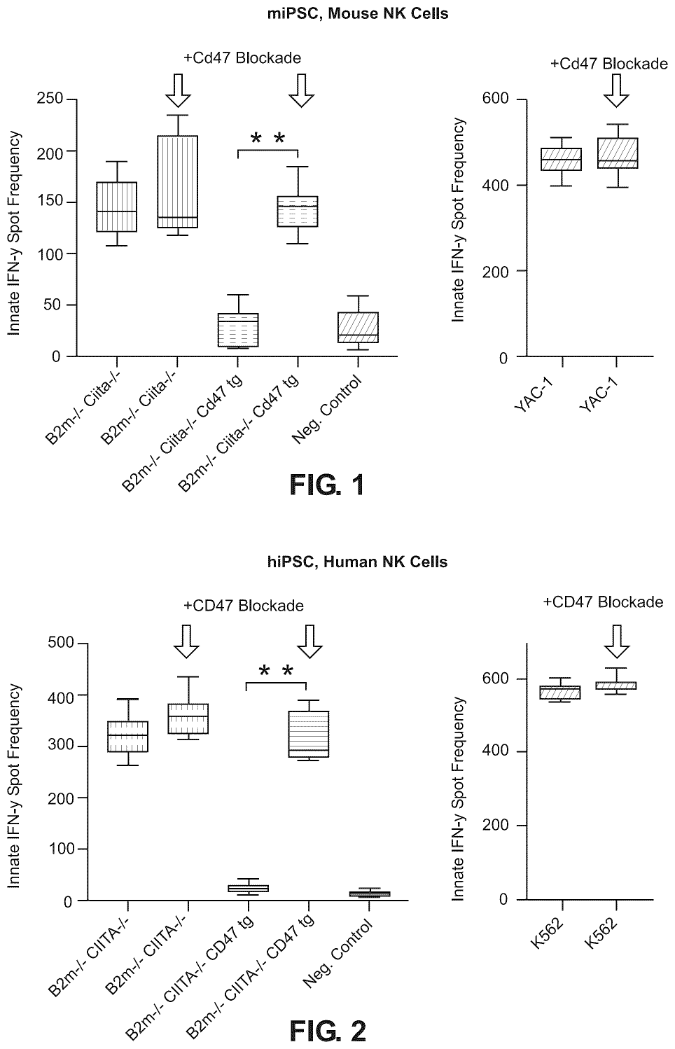

Mouse B2m−/−Ciita−/−CD47 tg iPSCs co-cultured with mouse NK cells such as approximately 95% NK cells and 5% macrophages failed to stimulate NK cell activation (FIG. 1). Mouse B2m−/−Ciita−/−iPSCs triggered IFNγ release by NK cells in the Elispot assay, while mouse B2m−/−Ciita−/−CD47 tg iPSCs did not. Blocking CD47 (e.g., use of an anti-CD47 antibody) had no effect on the mouse B2m−/−Ciita−/−iPSCs. However, CD47 blockage fully abolished the protection B2m−/−Ciita−/−CD47 tg iPSCs had. YAC-1 cells which are known to activate NK cells and thus release of IFNγ served as a control.

Human B2M−/−CIITA−/−CD47 tg iPSCs co-cultured with human NK cells also failed to stimulate NK cell activation. FIG. 2 shows that human B2M−/−CIITA−/−iPSCs triggered IFNγ release by NK cells in the Elispot assay, while human B2M−/−CIITA−/−CD47 tg iPSCs did not. Blockage of CD47 had no effect on human B2M−/−CIITA−/−iPSCs, but it did abolish the protection human B2M−/−CIITA−/−CD47 tg iPSCs had. K562 cells which are known to activate NK cells and thus release of IFNγ served as a control.

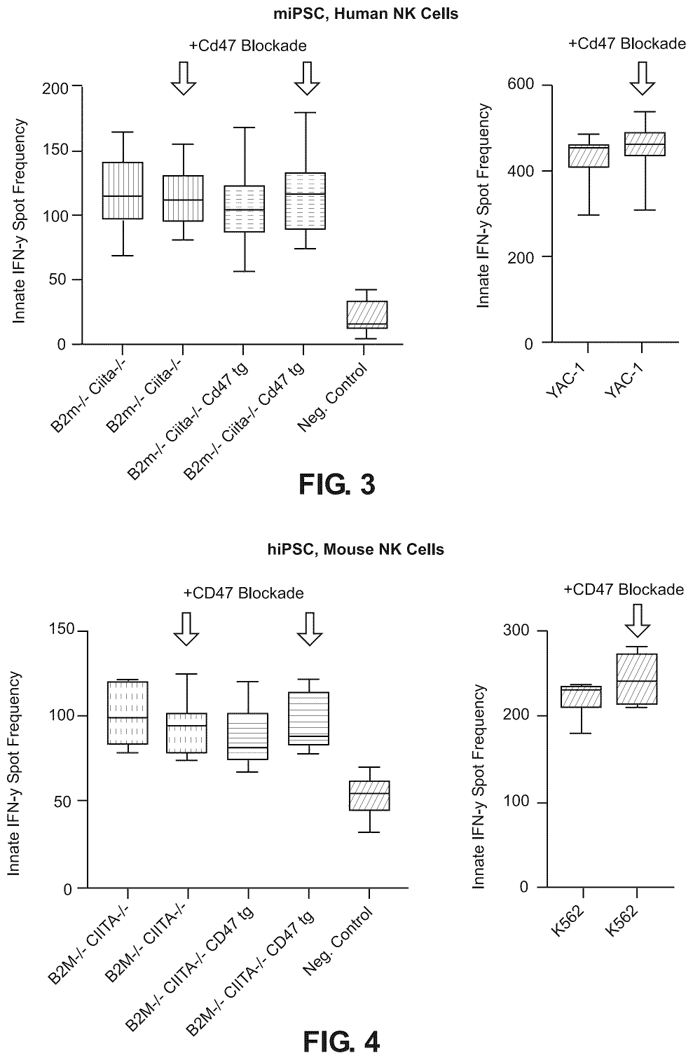

FIG. 3 shows Elispot results of mouse B2m−/−Ciita−/−CD47 tg iPSCs incubated with human NK cells (approximately 95% NK cells and 5% macrophages). Mouse B2m−/− Ciita−/−iPSCs and mouse B2m−/−Ciita−/−CD47 tg iPSCs triggered IFNγ release by human NK cells. Blockage of CD47 had no effect on the NK cell response. YAC-1 cells elicited a strong IFNγ release by human NK cells and served as a control.

FIG. 4 shows Elispot results of human B2M−/−CIITA−/−CD47 tg iPSCs incubated with mouse NK cells (approximately 95% NK cells and 5% macrophages). Human B2M−/−CIITA−/−iPSCs and human B2M−/−CIITA−/−CD47 tg iPSCs triggered IFNγ release by mouse NK cells. Blockage of CD47 had no effect on the NK cell response. Human K562 cells elicited a strong IFNγ release by mouse NK cells and served as a control.

Macrophage phagocytosis assays were also performed to determine if the HIP cells of the present invention are susceptible to macrophage phagocytosis. Briefly, HIP cells described herein were labeled with firefly luciferase and co-cultured with macrophages. The viability or presence of the HIP cells was analyzed by a luciferase reporter assay.

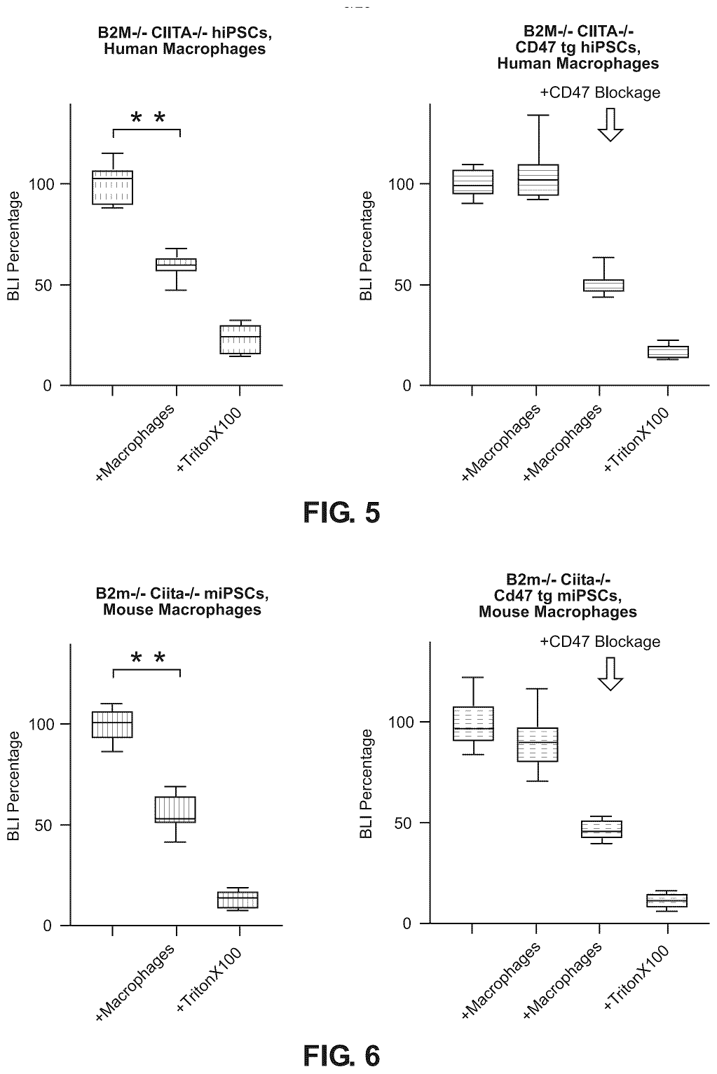

FIG. 5 shows phagocytosis assay results of firefly luciferase labeled human B2M−/−CIITA−/−CD47 tg iPSCs co-cultured with human macrophages. The viability signal of the human B2M−/−CIITA−/−iPSCs significantly dropped when incubated with macrophages. On the other hand, the viability signal of the human B2M−/−CIITA−/−CD47 tg iPSCs did not change in the presence of human macrophages. TritonX-100 which killed all HIP cells was used as a control. Blockage of CD47 eliminated the protective features of human B2M−/−CIITA−/−CD47 tg iPSCs and made them susceptible to macrophage phagocytosis or elimination.

FIG. 6 shows phagocytosis assay results of firefly luciferase labeled mouse B2m−/−Ciita−/−CD47 tg iPSCs co-cultured with mouse macrophages.

The viability signal of the mouse B2m−/−Ciita−/−iPSCs significantly dropped when incubated with macrophages. In contrast, the viability signal of the mouse B2m−/−Ciita−/−CD47 tg iPSCs did not change in the presence of mouse macrophages. TritonX-100 which killed all HIP cells was used as a control. Blockage of CD47 eliminated the protective features of mouse B2M−/−CIITA−/−CD47 tg iPSCs and made them susceptible to macrophage phagocytosis or elimination. TritonX-100 which killed all HIP cells was used as a control.

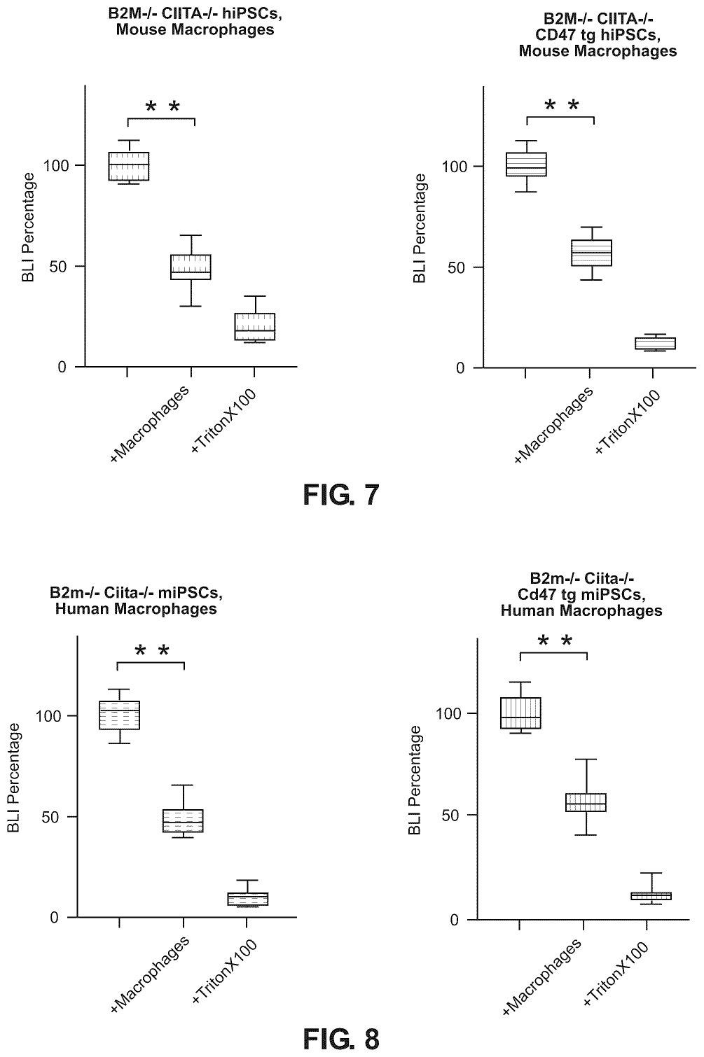

FIG. 7 shows phagocytosis assay results of firefly luciferase labeled human B2M−/−CIITA−/−CD47 tg iPSCs co-cultured with mouse macrophages. The viability signals of both human B2M−/−CIITA−/−iPSCs and human B2M−/−CIITA−/−CD47 tg iPSCs dropped significantly when co-cultured with mouse macrophages. TritonX-100 which killed all HIP cells was used as a control.

FIG. 8 shows phagocytosis assay results of firefly luciferase labeled mouse B2m−/−Ciita−/−CD47 tg iPSCs co-cultured with human macrophages. The viability signals of both mouse B2m−/−Ciita−/−iPSCs and mouse B2m−/−Ciita−/−CD47 tg iPSCs dropped significantly when co-cultured with human macrophages. TritonX-100 which killed all HIP cells was used as a control.

The results provided herein show that mouse B2m−/−Ciita−/−CD47 tg iPSCs and human B2M−/−CIITA−/−CD47 tg iPSCs were able to evade innate immune responses, such as NK cell activation and macrophage phagocytosis.

### Example 4: Generation of Human Induced Pluripotent Stem Cell Derived Cardiomyocytes

Provided herein are methods for generating human HIP cell-derived cardiomyocytes (CMs). In an exemplary embodiment, human HIP cells were differentiated into an in vitro monolayer of cardiomyocytes. The exemplary procedure described herein was adapted from Sharma et al., J Vis Exp, 2015, 97, doi: 10.3791/52628, hereby incorporated by reference in its entirety and specifically for the techniques to differentiate the cells.

Human HIP cells were plated on diluted Matrigel (356231, Corning) in 6-well plates and maintained in Essential 8 Flex media (Thermo Fisher). Media was changed every 24 hours.

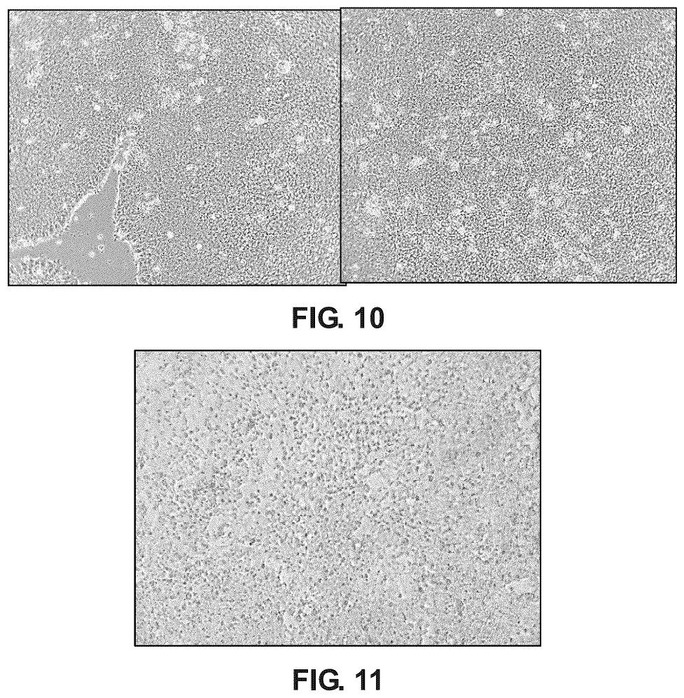

After the cells were at 90%-100% confluency, the differentiation was started (FIG. 10) and CM differentiation media at Day 0 was changed to 5 mL of RPMI1640 (Gibco, cat. no. 61870) containing 2% B-27 minus Insulin (Gibco, cat. no. A1895601) and 2 μM-8 μM CHIR-99021 (Selleck Chem, cat. no. S1263). In some cases, the concentration of CHIR-99021 was 6 μM. There were no media changes between Day 0 and Day 2 of differentiation.

On Day 2 (FIG. 11), CM differentiation media was changed to RPMI1640 containing 2% B-27 minus insulin without CHIR-99021. Care was taken not to agitate the cells.

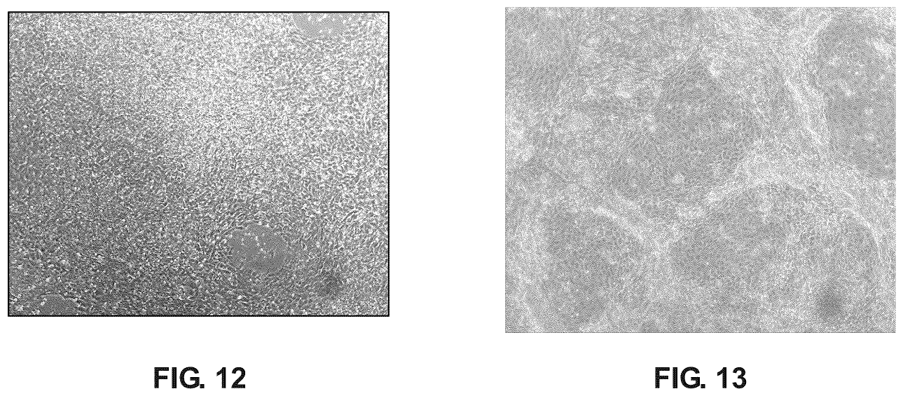

On Day 3 (FIG. 12), CM differentiation media was changed to RPMI1640 containing 2% B-27 minus insulin and 5 μL IWR1 (a WNT inhibitor, Selleck Chem, cat. no. S7086). Care was taken not to agitate the cells. After the media was changed, the cells were allowed to remain undisturbed in the 37° C. incubator for 48 hours. There were no media changes between Day 3 and Day 5 of differentiation.

On Day 5 (FIG. 13), CM differentiation media was changed back to RPMI1640 media containing 2% B-27 minus insulin and incubated for 48 hr. The CM differentiation media on Day 5 was the same as the media on Day 2. After the media change, the cells were allowed to remain undisturbed in the 37° C. incubator for 48 hours.

At Day 7 (FIG. 14), CM differentiation media was changed to RPMI1640 containing B27 plus insulin (Gibco, cat. no. 17504044) and replaced every other day thereafter with the same media. Spontaneous beating of cardiomyocytes was first visible at approximately day 8 to day 10.

After 10 day of differentiation (FIG. 15), more than 50% of the cell areas were beating. Branch-like structures were highly pronounced. In some cases, cells were terminally differentiated and no longer proliferating. Since the cells were beating, the culture of cardiomyocytes was maintained in Day 7 CM differentiation media. Media was changed every other day.

In some examples, when the differentiated cells were not beating or if there were small beating areas, the cells were subjected to glucose starvation. Glucose starvation loosened non-cardiomyocytes cells from the culture plate. The media was changed at differentiation day 10 to purification media comprising RPMI1640 without glucose (Gibco, cat. no. 11879) containing B-27 plus insulin (Gibco, cat. no. 17504044). The cells were maintained in purification media for 3 days (until day 13 of differentiation).

At Day 13, media was changed to Day 7 CM differentiation media to maintain the cardiomyocytes in the culture. In some examples, the purification procedure (glucose starvation) was repeated on Day 14 such that the media was changed to CM differentiation media on Day 17. The remaining cells were highly purified cardiomyocytes.

The isolated and purified cardiomyocytes were maintained in Day 7 CM differentiation media of Day 7. The media was changed every other day. In some embodiments, about 1×10⁶ cardiomyocytes were plated in one 6-well plate.

Beating cardiomyocytes were frozen in freezing media and stored in liquid nitrogen. In some cases, the freezing media included 90% heat-inactivated FCS and 10% DMCO, or a xeno-free equivalent. After thawing, the cardiomyocytes continued to beat.

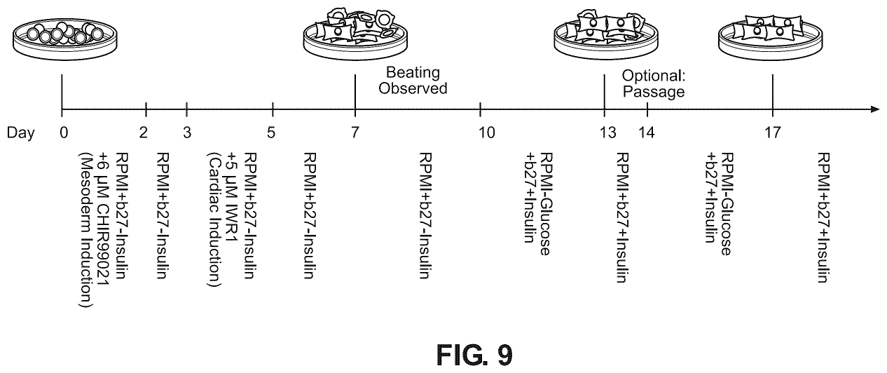

FIG. 9 provides a diagram of the differentiation method. FIG. 10 shows human iPSCs cultured on Matrigel™ immediately before starting the differentiation (100× magnification). FIG. 11 shows cells on differentiation day 2 before media change (100× magnification). The cells remained in a monolayer with approximately 10% floating cells. The differentiation media was yellowish prior to the media change. FIG. 12 shows cells on differentiation day 3 before media change (100× magnification). The cells remained in a monolayer with approximately 20% floating cells. The media was less yellowish in color compared to differentiation day 2. Some vesicle-like structures were visible in the monolayer. FIG. 13 shows cells on differentiation day 5 before media change (100× magnification). The cells remained in a monolayer with approximately 15% floating cells. Cardiomyocytes appeared above other cell clusters. FIG. 14 shows cells on differentiation day 7 before media change (100× magnification). The cells remained in a monolayer with approximately 15% floating cells. Branch-like structures were visible. FIG. 15 shows cells on differentiation day 9 before media change (100× magnification). Beating cardiomyocytes were visible and appeared darker in the photo.

### Example 5: Murine HIP Cells Differentiated into Induced Cardiomyocytes

mHIP cells were differentiated into murine induced cardiomyocytes (hmiCM). Prior to differentiation, mHIP cells and miPSCs were passaged two times on gelatin-coated dishes to remove the feeder cells. At day 0, differentiation was started with 80,000 cells/mL in IMEM/Ham's F12 (3:1, both Corning)+0.5% N2-Supplement, 1% B27 retinoic acid, 0.05% BSA, 1% pen-strep, 1% glutamine (Gibco), 5 mg/mL ascorbic acid and 40 nL/mL MTG (both Sigma-Aldrich) for 2 days in uncoated 10 cm plates. At day 2, cells were transferred in IMEM/Ham's F12 (3:1, both Corning) with 0.5% N2-Supplement, 1% B27 retinoic acid, 0.05% BSA, 1% pen-strep, 1% glutamine (all Gibco), 5 mg/mL ascorbic acid and 40 nL/mL MTG (Sigma-Aldrich) for 2 days in uncoated 10 cm plates. On day 4, cells were plated in gelatin-coated 6-well plates in SP34 media containing 1% glutamine, 50 μg/mL ascorbic acid, 5 ng/mL VEGF, 500 μg/mL hFGFb, and 25 ng/ml hFGF10 (R&D Systems). Media was changed on day 7 to SP34 media containing 1% glutamine and 50 μg/mL ascorbic acid and was changed every other day. Cell Beating started around day 11-14 and demonstrated their function.

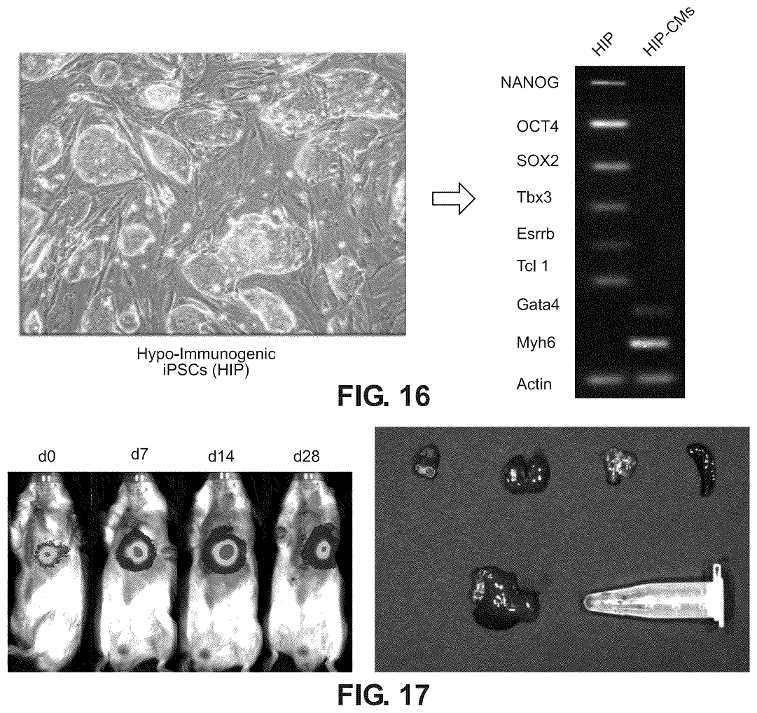

The cells were enriched by separation using MACS according the manufactures' protocol using anti-CD15 mAb-coated magnetic microbeads (Miltenyi) for negative selection. The flow-through containing enriched hiCMs and miCMs were re-plated and used for different assays. Differentiation was confirmed by rtPCT for Gata4 and Mhy6 (FIG. 16). The Gata4 forward primer (SEQ ID NO:7 was:

5′-CTGTCATCTCACTATGGGCA-3′

and the reverse primer (SEQ ID NO:8 was:

5′-CCAAGTCCGAGCAGGAATTT-3′

The Mhy6 forward primer (SEQ ID NO:9) was

5′-ATCATTCCCAACGAGCGAAAG-3′

and the reverse primer (SEQ ID NO: 10) was

5′-AAGTCCCCATAGAGAATGCGG-3′

Differentiation was also confirmed by immunofluorescence (IF). The primary antibodies were against α-sarcomeric actinin (EA-53, Abcam) or troponin I (ab47003, Abcam) followed by the corresponding secondary antibody conjugated with AF488 or AF555 (Invitrogen). (Data not shown.)

### Example 6: Transplanted Hypo-mCMs Survive Long-Term in Allogeneic Recipients

hiCMs and miCMs were transplanted into the infarct border zone of allogeneic recipient mice (BALB/c). The cell line are luciferase (+) that generated luc (+) CMs that were followed in vivo by bioluminescence (BLI).

HIP cells and iPSCs Transduction to express luciferase. The cells were transduced to express Fluc. One hundred thousand mHIP cells or miPSCs were plated in gelatin coated 6-well plates and incubated overnight at 37° C. and 5% CO₂. The next day, the media was changed and one vial of Fluc lentiviral particles expressing the luciferase II gene under a re-engineered EF1a promotor (GenTarget, San Diego, CA) was added to 1.5 ml media. After 36 hours, 1 ml of culture media was added. After another 24 hours, a complete media change was performed. After 2 days, luciferase expression was confirmed by adding D-luciferin (Promega, Madison, WI). Signals were quantified with an IVIS 200 (Perkin Elmer Waltham, MA) in maximum photons s⁻¹ cm⁻² per steradian.

The hiCMs were not rejected and did not migrate into other organs 28 days post-transplantion (FIG. 17). The hiCMs evaded immune recognition after allogeneic transplantation showing longitudinal survival. Because injection was into the cardiac muscle, the transplanted cells were optically mapped. hiCMs resulted in “supercell” engraftment but none with the miCMs. (Data not shown.)

### Optical Mapping:

Whole-Heart Langendorff Preparation. Four to five weeks after surgery, mice were euthanized by cervical dislocation. The hears were quickly excised and placed in ice-cold modified Tyrode's solution of composition (in mmol/L) 93 NaCl, 20 NaHCO₃, 1 Na2HPO4, 1 MgSO4, 5 KCl, 1.8 CaCl2, 20 Na-acetate, 20 glucose. Hearts were mounted via the aorta onto a cannula and retrogradely perfused at 9 ml/min using the same Tyrode's solution at 37° C. with pH maintained at 7.4 by bubbling with a 95% O₂/5% CO₂ gas mixture. Perfusate was then switched to a Tyrode's solution containing 10 mmol/L 2,3-butanedione monoxime (BDM) and 10 μmol/L blebbistatin (Enzo Life Sciences, Exeter, United Kingdom) to inhibit contraction and minimize movement artifacts. The hearts were positioned horizontally in a custom-built Perspex chamber to enable imaging of the left ventricle (LV). A pseudo-electrocardiogram (ECG) was monitored throughout the experiment using 2 Ag/AgCl disk electrodes placed close to the heart, with a reference electrode placed in the perfusion bath. Two platinum electrodes connected to an isolated stimulator were positioned on the right atrial (RA) appendage to enable pacing of the hearts via the physiological endocardium to epicardium conduction pathway, at a cycle length of 200 ms (5 Hz).

Optical measurements were made as described in detail previously (Kelly et al., Circ. Arrhythmia Electrophysiol 6:809-17 (2013), incorporated herein by referenced in its entirety.) Briefly, hearts were loaded with a 50 μL bolus of 2 mM voltage-sensitive dye (di-4-ANEPPS) over a 10 min period. Widefield single-photon epifluorescence recordings from the LV were made using a CardioCMOS-SM128 camera (Redshirt Imaging. Decatur, GA) with a 590 nm long pass emission filter. Excitation was provided by LED light at 470 nm. Image resolution was 128×128 pixels/frame, and recordings were made at a frame rate of 2.5 kHz. Two photon (2P) laser scanning microscopy (2PLSM) was carried out using a Zeiss LSM 510 NLO upright microscope (Carl Zeiss, Jena, Germany) equipped with a Ti:Sapphire 690-1080 nm tunable laser (Chameleon Ultra II, Coherent, Santa Clara, CA). These 2P measurements provided a high degree of depth resolution, enabling identification of the discrete tissue layers exhibiting electrical activity (Rubart, 2004). Di-4-ANEPPS was excited at 920 nm, with emission collected by two bi-alkali PMT detectors at 510-560 nm and 590-650 nm, respectively, enabling ratiometric measurements to be made. Line scans, with a scan time of 0.39 ms for short scans and 1.93 ms for long scans, were performed in the direction of cell orientation observed at the epicardial surface. Line scanning was initiated following the arrival of a trigger pulse, synchronized by the electrical stimulus pulse used to pace the hearts.

Sequential widefield and 2P voltage recordings were made in myocardium remote from the infarct scar in the NZ, in the BZ at the edge of the visible scar, adjacent to the NZ, and within the IZ toward the center of the scar. Widefield electrical mapping using a 10×/0.3 NA objective lens (Carl Zeiss, Jena, Germany) was first used to identify the areas of remnant electrical activity within the BZ and IZ. Widefield electrical mapping was then repeated on regions displaying electrical signals with a 40×/0.8 NA objective (Carl Zeiss, Jena, Germany) to further localize electrically active areas. Finally, structures in the optical plane were imaged using 2P excitation in frame scan mode, then electrical signals recorded using line scan mode at discrete depths below the epicardial surface. A series of recordings were made, starting at 50 μm below the surface and at increasing depths (50-100 μm steps) until the signal-to-noise ratio (S/N) became too low to distinguish a clear action potential (AP) signal. The ability to visualize clear structures decreased at deeper layers: the maximum depth from which discernible images could be obtained depended on the zone, but electrical signals from line scan recordings could always be recorded beyond the layers where structures could be imaged. It was therefore not possible to identify the source of electrical activity directly from images of tissue structure at the deeper layers. A subset of analyses was performed using a modified upright 2P laser scanning setup (Intelligent Imaging Innovations; Denver, CO) utilizing a pair of high sensitivity GaAsP PMT detectors, and using a combination of FluoVolt and Rhod2AM, excited at 840 nm. These were used to verify findings in a higher sensitivity setup.

Determining the Source of Electrical Activity in the Scar. To determine the cellular origin of electrical activity in the scar, measurements of intracellular Ca²⁺ were made in regions where voltage signals were measured by prior loading of the myocardium with Fura-2/AM. If the signals were arising from residual myocytes Ca²⁺ transients (CaTs) would be expected in response to an electrical stimulus. However, if the voltage signals were arising from abnormal myocytes or other cellular entities (i.e., fibroblasts or myofibroblasts), CaTs may not be produced in response to electrical stimulation (Chilton et al. 2007).

Tyrode's solution for these experiments was supplemented with 1 mmol/L probenecid to block anion transporters that excrete Fura-2, thus improving dye retention in the cell. (Di Virgilio et al., Biochem. J. 256:959-63 (1988), incorporated by reference herein in its entirety.) Fura-2/AM was prepared as a 1 mmol/L stock in DMSO-pluronic acid F-127 (25% w/v). A 100 μL bolus of dye was injected into a bubble trap in the perfusion line to allow dilution of the dye and slow loading into the heart. Additional boluses of dye were injected if required due to low or time-dependent loss of fluorescence signal. Fura-2/AM was 2P excited at 760 nm, and fluorescence emission was directed through a short-pass 650 nm dichroic mirror and collected at 510-560 nm. Ca²⁺ measurements were made immediately after any voltage signals were detected in the same plane of focus and using the same scan line and 1.93 ms scan time.

Data Analysis and Interpretation. Widefield voltage signals were averaged from 3×3 pixel arrays. 2P voltage and Ca²⁺ signals were processed using custom written software that utilized the information from exact cycle length times and line scan rates to align and produce an averaged voltage or Ca²⁺ trace from 25 sequential stimuli (5 s of recordings). AP signal characteristics were analyzed from the averaged trace. These measurements included 10-90% upstroke rise time (TRise) and duration of the AP from 50% activation to 50, 75, and 90% repolarization (APD50, APD75, and APD90, respectively). Activation times were determined as the time of arrival of the AP at that point in the LV wall relative to the time of stimulation.

S/N was calculated as the peak amplitude of the whole trace following a single stimulus pulse (signal) divided by the peak amplitude of the trace during the diastolic period (noise) (Supplementary Figure S1). An S/N value of 1 indicated no signal over and above the noise of the baseline. Based on observations of individual traces and corresponding S/N values, an S/N>1.4, was considered a meaningful transient signal (voltage or Ca2+). All traces with S/N>1.4 were further scrutinized to rule out artefactual signals produced by movement or noise spikes. All data are expressed as mean±standard error. Groups of data were compared using Student's t-test.

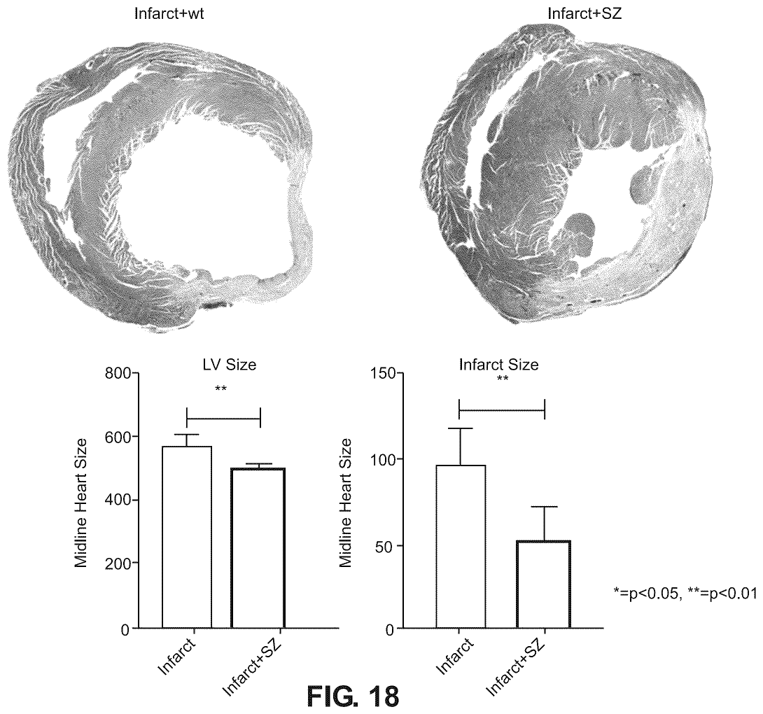

Histopathology and trichrome staining of recipient hearts 28 days after myocardial infarction revealed that the infarct size in allogeneic recipients of hiCMs was significantly reduced as well as the size of the left ventricle. Infarct length was measured from 25 slides with a gap of 150 μm each (every 10th slide, with 3 sections a 5 μm on each slide: slide 1-250). We identified alpha-sarcomeric positive donor cardiomyocytes and premature vessel structures in the recipients receiving the hiCMs cells. In contrast no cells were detected with the allogeneic miCMs (FIG. 18).

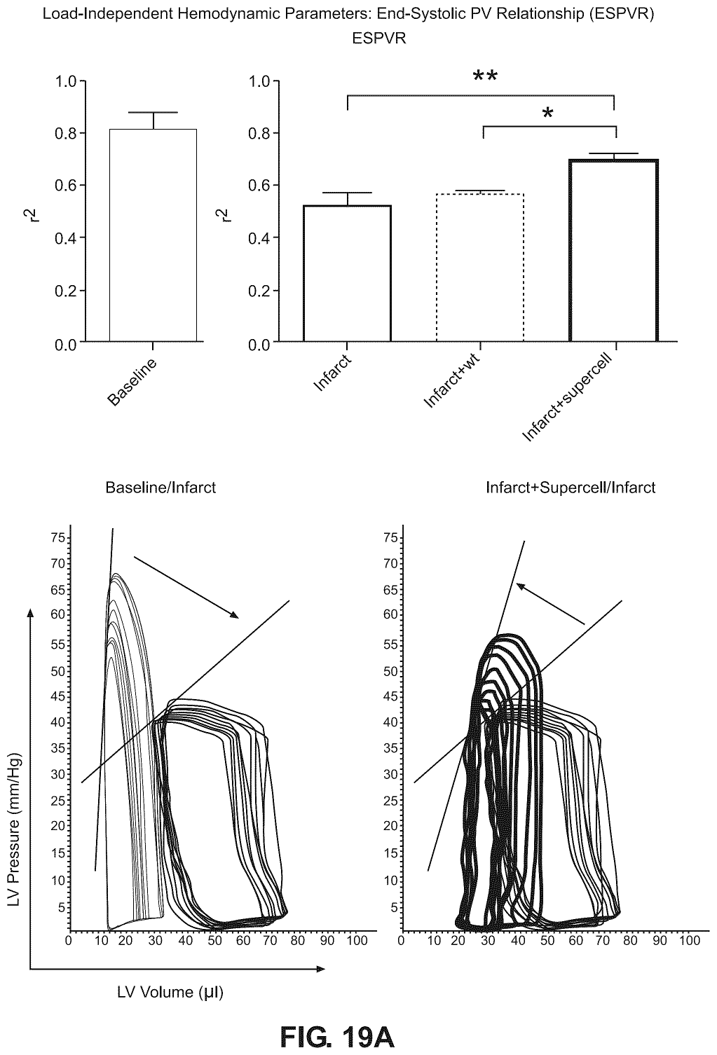

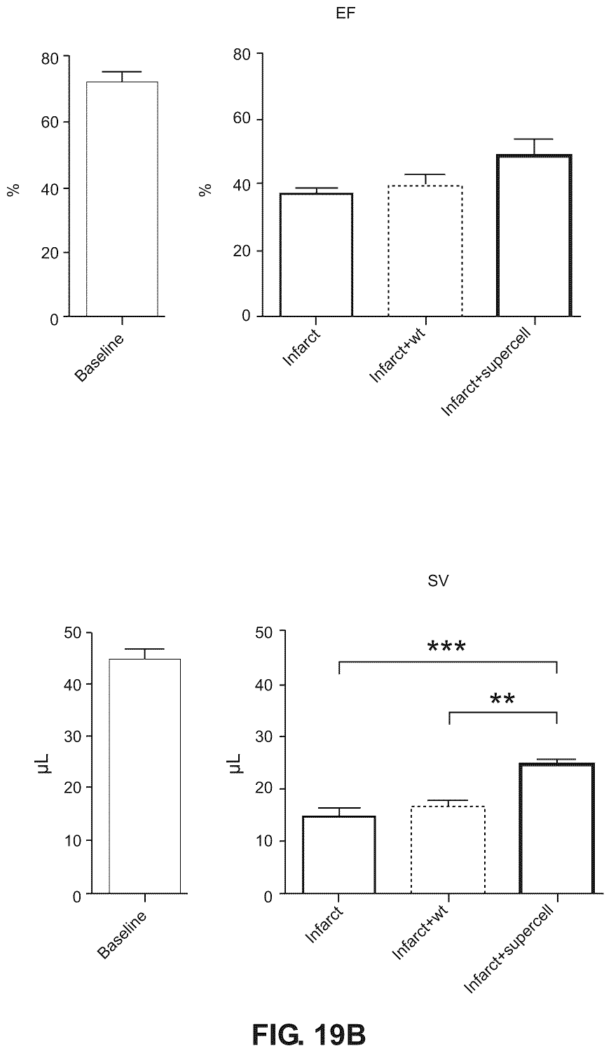

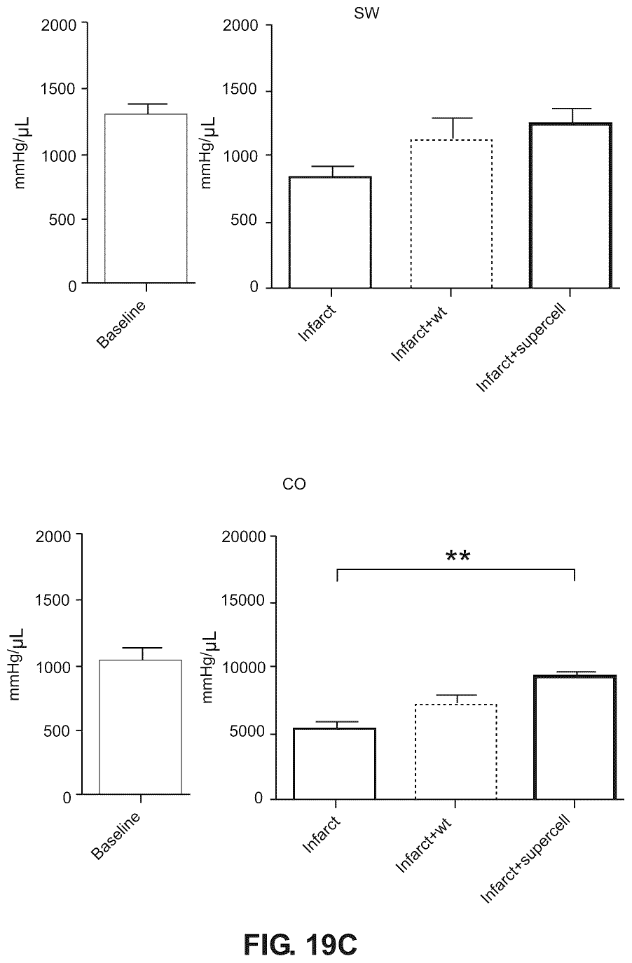

Detailed PV-loop analysis revealed a significant improvement of left-ventricular parameters. IT indicated not only survival of allogeneic cells, but also that they were engrafted and functionally restored the heart after myocardial infarction (FIG. 19A). PV-loop analysis of infarct hearts receiving either miCMs (green) or hiCMs “supercell” (red) injections.

The following parameters showed that the hiCMs restored heart function: Ejection fraction (EF) is the ratio of the volume of blood ejected from the ventricle per beat to the volume of blood in that ventricle at the end of diastole. Stroke volume (SV) is the volume of blood ejected by a ventricle in a single contraction. It is the difference between the end diastolic volume and the end systolic volume. (FIG. 19B.) Ventricular stroke work (SW) is defined as the work performed by the left ventricle to eject the stroke volume into the aorta. Cardiac output (CO) is defined as the amount of blood pumped by the ventricle in unit time. (FIG. 19C.) End-systolic pressure volume relationship (ESPVR) describes the maximal pressure that can be developed by the ventricle at any given cardiac chamber volume. The ESPVR is relatively insensitive to changes in preload, afterload, and heart rate. This makes it an improved index of systolic function over other hemodynamic parameters like ejection fraction, cardiac output, and stroke volume.

Myocardial infarction results in reduced pump function of the heart, with increased volume and decreased pressure in the P-V-loop analysis. When HIP-cardiomyocytes were injected after myocardial infraction, the changes in pressure and volume are prevented, indicating regeneration of the heart and prevented remodeling.

### Example 7: Human HIP Cells Differentiated into hiCMs

Human HIP cells were differentiated into hypoimmune cardiomyocytes. hHIP cells were plated on diluted Matrigel (356231, Corning) in 6-well plates and maintained in Essential 8 Flex media (Thermo Fisher). Differentiation was started at 90% confluency, and media was changed to 5 mL of RPMI-1640 containing 2% B-27 minus Insulin (Gibco) and 6 μM CHIR-99021 (Selleckchem). After 2 days, media was changed to RPMI-1640 containing 2% B-27 minus insulin without CHIR. On day 3, 5 μL IWR1 was added to the media for two further days. At day 5, the media was changed back to RPMI-1640 containing 2% B-27 minus insulin medium and left for 48 hr. At day 7, media was changed to RPMI-1640 containing B27 plus insulin (Gibco) and replaced every 3 days thereafter with the same media.

Purification of cardiomyocytes was performed on day 10 post-differentiation. Briefly, media was changed to low glucose media and maintained for 3 days. At day 13, media was changed back to RPMI-1640 containing B27 plus insulin. This procedure was repeated on day 14 again.

The differentiation phenotype was confirmed by rtPCR for troponin (cTNT, data not shown). The forward primer (SEQ ID NO:11) was:

5′-GAGGCACCAAGTTGGGCATGAACG A-3′,

The reverse primer (SEQ ID NO:12) was:

5′-GGCAGCGGAAGAGGATGCTGAA′

The differentiation phenotype was also confirmed by immunofluorescence (IF) staining. The primary antibodies were against α-sarcomeric actinin (EA-53, Abcam) and troponin I (ab47003, Abcam), followed by the corresponding secondary antibody conjugated with AF488 or AF555 (Invitrogen). Cell nuclei were stained with DAPI. Imaging was performed using a Leica SP5 laser confocal microscope (Leica). (Data not shown.)

Spontaneous beating of cardiomyocytes was first visible around day 10 of differentiation. The cardiomyocytes show similar beating rates to human cardiac tissue and are sensitive to cardiac stimulation (by isoprenaline) and cardiac blocking (by verapamil).

### Example 8: hiCM Cells Survive Allogenic Transplantation in Humanized Mice

Hypoimmune cardiomyocytes survived following transplantation into allogeneic humanized mice (Humanized NSG-SGM3 mice (18-30 weeks) were purchased from Jackson Laboratories (Sacramento, CA). Human CD34+ hematopoietic stem cell-engrafted NSG-SGM3 mice develop multi-lineage human immune cells, and demonstrate a functional human immune system displaying T cell-dependent immune responses with no donor cell immune reactivity towards the host. Animals were randomly assigned to experimental groups. The percentage of CD3+ cells among the human CD45+ cell population was assessed in every animal and CD3 percentages were never significantly different between WT and B2M−/−CIITA−/−CD47 tg groups (Deuse et al., Nat Biotech 37 (3): 252-258 (2019), incorporated by reference herein in its entirety.)

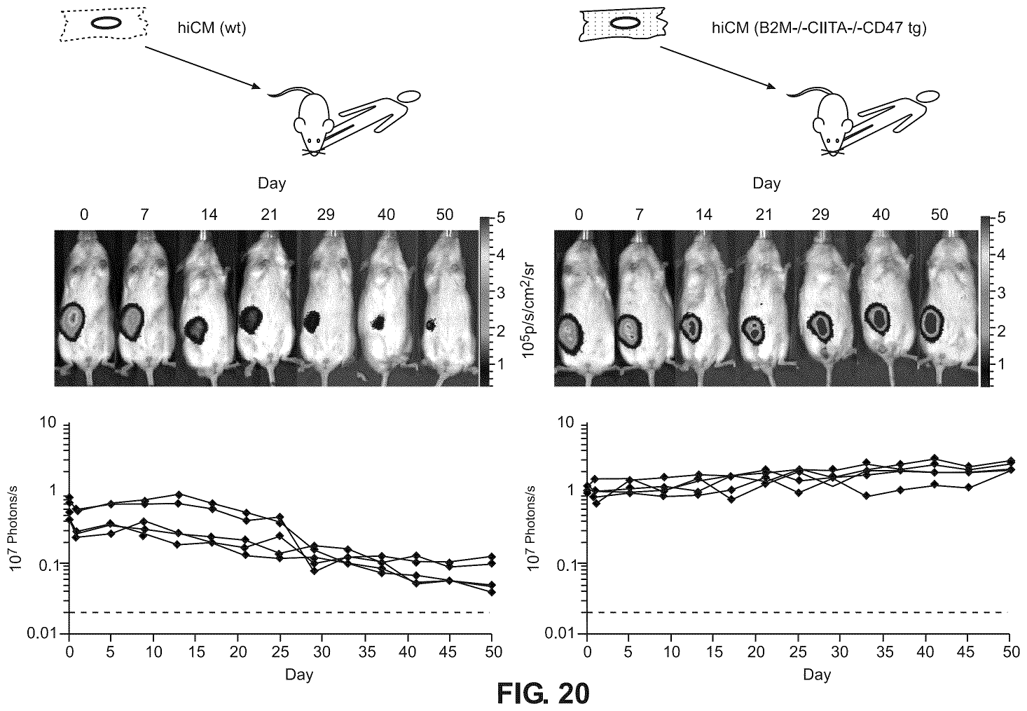

Wild-type or B2M−/−CIITA−/−CD47 tg hiCMs transplanted intramuscularly into allogeneic humanized mice confirmed the mouse data in an additional humanized mouse model. Cardiomyocytes were longitudinally followed by bioluminescence (BLI). All wt hiCM grafts were rejected over time (5 animals). All of the five B2M−/−CIITA−/−CD47 tg hiCM grafts permanently survived (FIG. 20). The hypoimmune hiCM cells also survived following transplantation after myocardial infarction (FIG. 21.)

### Example 9: Generation of Mouse Induced Pluripotent Stem Cell Derived Endothelial Cells

Provided herein are methods for generating human iPSC derived endothelial cells (ECs). In an exemplary embodiment, murine iPSCs (such as murine HIP cells) can be differentiated into an in vitro monolayer of endothelial cells.

The protocol includes using a 6-well format plate or a 10-cm dish. Cells were trypsinized with standard trypsin. However, the trypsinized cells were not centrifuged. Rather, the trypsinized colonies were resuspended in media at a volume greater than 3 times the volume of trypsin used.

Before differentiation (approximately 12 days), mouse embryonic fibroblasts (MEFs) were thawed-1×10⁶ MEF per well (6-well plate) on gelatin coated plates using MEF media. 1 vial of iPSCs were thawed and transferred into three 6-wells on MEFs. ESC media was changed every day. Colonies were split on MEF 1:3-1:6 in ESC media (approximately 8 days after thawing). NT-ESC colonies were observed on MEFs before splitting (100× magnification). Colonies were split from MEFs onto gelatin 1:3-1:6 in ESC media (approximately 4 days after prior splitting; also referred to as Day-2).

On Day 0-Day 2, cells were 70-85% confluent to start the differentiation protocol. The EC differentiation media #1 included RPMI and B-27 supplement minus insulin (Life Technologies) and 5 μM CHIR-99021 (Selleck Chemicals, Houston, TX, USA) for 2 days. About 2.5 ml media per 6-well plate or 10 cm plate was used. No media was changed between day 0-2. The cells were left in the incubator without moving.

On Day 2-Day 4, the cell culture morphology was observed. The media was changed to EC differentiation media #2 containing RPMI and B-27 supplement minus insulin and including 2 μM CHIR-99021. Care was taken to avoid agitating the cells.

On Day 4-Day 7, endothelial cell (EC) differentiation media #3 comprising RPMI media minus insulin was used. Media changes were performed on day 4 and day 6. Undifferentiated cell clusters remained floating and the initial EC colonies appeared.

On Day 7-Day 17, the EC colonies continued to grow to confluence in the plate. Endothelial cell (EC) differentiation media #3 comprising EC media (Lonza, Benicia, California) supplemented with VEGF, FGF, ROCK inhibitor, SB431542, and Y-27632. During EC differentiation, media was changed about every other day (such as on day 7, day 9, day 11, and day 13).

### Materials and Reagents

Trypsin: Gibco, Cat. No. 12605-010.
Gelatin coating: EmbryoMax® 0.1% Gelatin Solution, Cat. No. ES-006-B. The surface of the plates were covered with the solution and the coated plates were stored at 37° C. until use.
MEF media: DMEM+glutamax+sodium pyruvate, with 4.5 g/L glucose (Gibco), 15% FBS, NEAA, and 1% Pencillin/Streptomycin (P/S).
EC media for Day 0-Day 2: RPMI and B-27 supplement minus insulin (Life Technologies Catalog number: A1895601, 5 M CHIR-99021 (Selleck Chemicals, Houston, TX, USA. Catalog No. S2924), and 1% P/S.
EC media for Day 2-Day 4: RPMI and B-27 supplement minus insulin (Life Technologies Catalog number: A1895601, 2 μM CHIR-99021 (Selleck Chemicals, Houston, TX, USA. Catalog No. S2924), and 1% P/S.
CHIR stock solution (10 mM) was diluted in media to 5 μM (1:2000) for Day 0-Day 2 media. CHIR stock solution was diluted in media to 2 μM (1:5000) for Day 2-Day 4 media.
EC media for Day 4-Day 7 (RPMI media minus insulin): RPMI and B-27 supplement minus insulin with 50 ng/mL vascular endothelial growth factor (VEGF; R&D Systems, Minneapolis, MN, USA), 10 ng/ml fibroblast growth factor basic (FGFb; R&D Systems), 10 μM Y-27632 (ROCK inhibitor) (Sigma-Aldrich, Saint Louis, MO, USA), and 1 μM SB 431542 (Sigma-Aldrich) for 3 days.

EC media for Day 7-Day 14 (EC media from Lonza): EGM-2 SingleQuots media (or CC-3162 EGM™-2 BulletKit™, EBM™-2 plus SingleQuots™ of Growth Supplements, 500 ml), 10 μM Y-27632 (Sigma-Aldrich, Saint Louis, MO, USA), 1 μM SB 431542 (Sigma-Aldrich), 25 ng/ml VEGF and 2 ng/ml FGF.

CD31+ cells were sorted and selected from murine ECs derived from pluripotent stem cells. Magnetic bead-based sorting methods including MACS was performed. For example, CD31 microbeads (Miltenyi, cat. No. 130-097-418) were used for positive selection of CD31+ cells. Cells after day 12-day 14 and prior to MACS sorting (100× magnification) were observed. EC colonies were 80%-90% confluent and undifferentiated cell clusters were present. Spindle-shaped, long cells represented the mesenchymal differentiated cells.

An illustrative protocol for MACS selecting of CD31+ cells is as follows.
1. Washed cells 1× with PBS
2. Trypsin: 0.5 ml/well; 5 min incubation at 37° C.
3. Stopped trypsinization with media (day 7-14 media), 1 ml/well
4. Used 1000 μl-Eppendorf pipet: resuspended well to dissolve clumps
5. Used yellow 100 μm mesh filter in 50 ml falcon tube to remove clumps and collected cells in 50 ml tube
6. Spun 50 ml tube at 300 g, 5 min, 4° C.
7. Looked at pellet in 50 ml tube. Big pellet=app. 90 Mio cells
8. Calculated per 10 Mio cells: 90 μl MACS buffer+10 μl beads
9. Removed supernatant to obtain a dry cell pellet
10. Resuspended pellet using a 1000 μl Eppendorf pipet in MACS buffer
11. Added with yellow 200 μl Eppendorf pipet beads to 10. And resuspended.
12. Vortexed (very short)
13. Incubated at 4° C. (fridge) for 30 min
14. Washed with 1-2 ml MACS buffer per 10 Mio cells by adding the buffer into the 50 ml tube
15. Centrifuged at 300 g, 5 min, at 4° C.
16. Placed MidiMACS columns into magnet. Added 3 ml MACS buffer into column (as “priming”). Under column: 50 ml Falcon tube
17. Removed supernatant from cell pellet (pellet needs to be dry)
18. Added 500 μl MACS buffer on pellet (always use 500 μl independent from pellet size)
19. Added the 500 μl (cells+MACS buffer) into column and waited until it passed the column
20. Added 3 ml pure MACS buffer into column and waited until it passed the column: performed 3 times (wash step)
21. Removed column from magnet and placed into 15 ml falcon tube
22. Added 5 ml MACS buffer into column and use “stamp” or “plunger”: Pressed the 5 ml into the 15 ml falcon tube to elute CD31+ cells
23. Spun cells at 300 g, 5 min, 4° C.
24. Pelleted (approximately 5-10Mio if big): plated into 1×6-well plate (gelatin coated)
25. Media: EC Differentiation media for day 7-day 14. Did not move plate before day 3 after MACS: do not take them out of the incubator before that.
26. Maintained differentiation media for day 7-14 until day 5.
27. Changed media on day 5 after MACS sorting to: Lonza EGM-2 SingleQuots media (CC-3162 EGM™-2 BulletKit™, EBM™-2 plus SingleQuots™ of Growth Supplements, 500 ml) plus 10 μM Y-27632.
28. Changed media every other day. Maintained cells on gelatin-coated plates.
29. When needed, cells were split 1:3 with trypsin.
30. To characterize the ECs, immunocytochemistry, LDL assays, and/or tube formation assays were performed according to methods known to those skilled in the art.

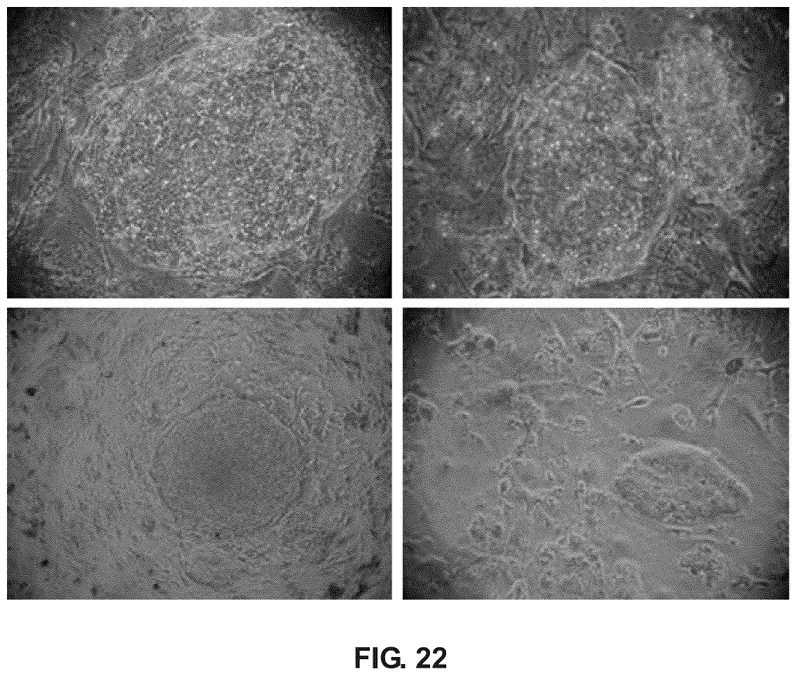

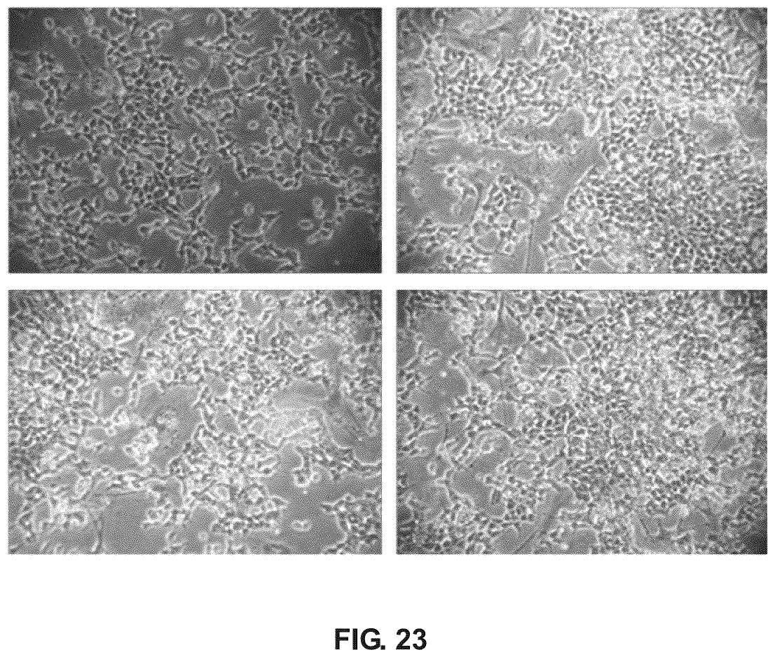

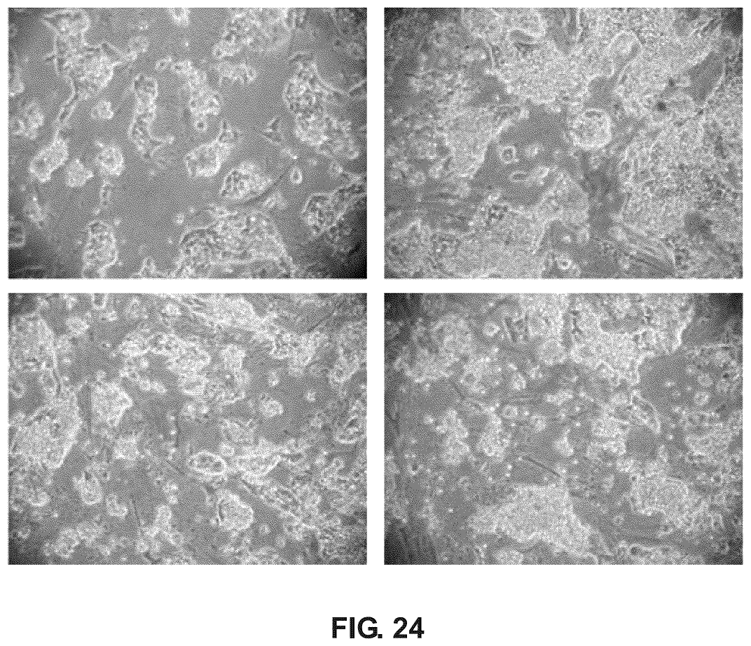

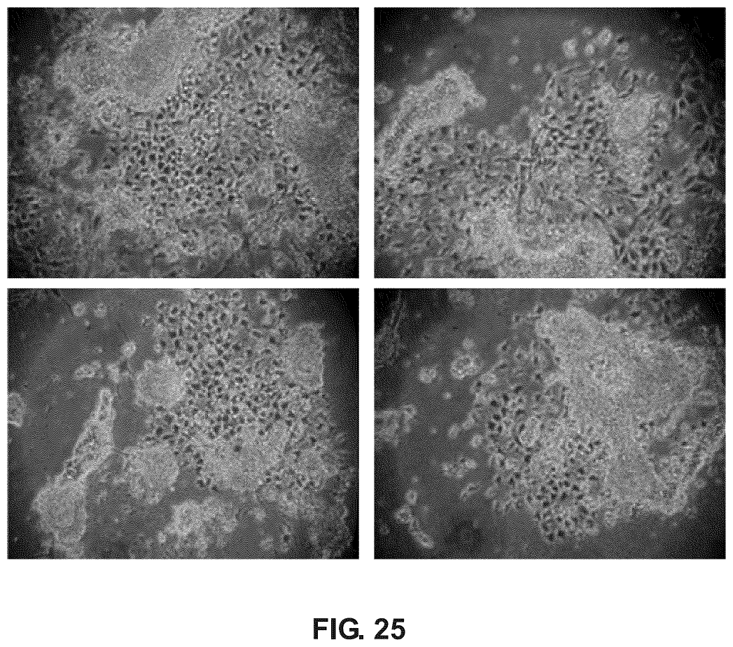

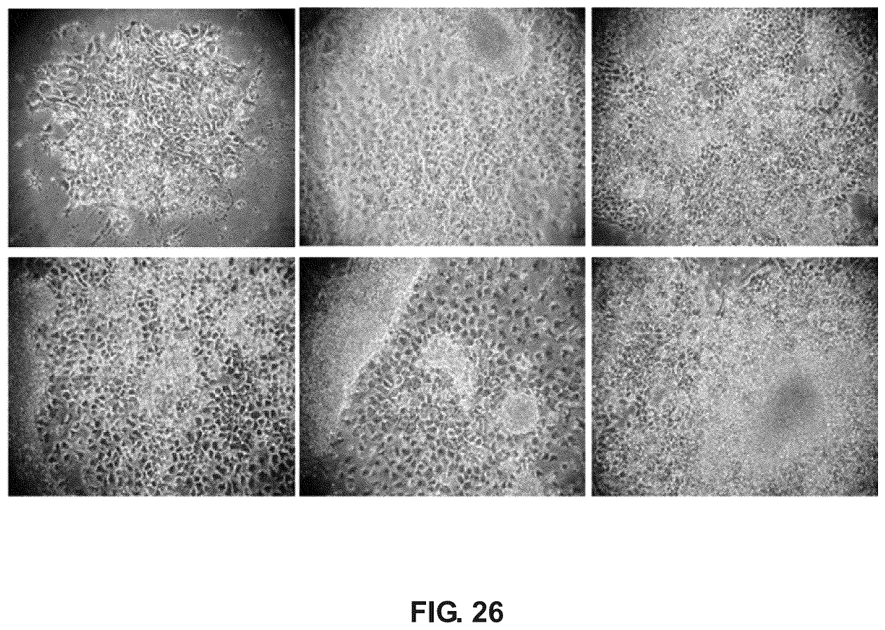

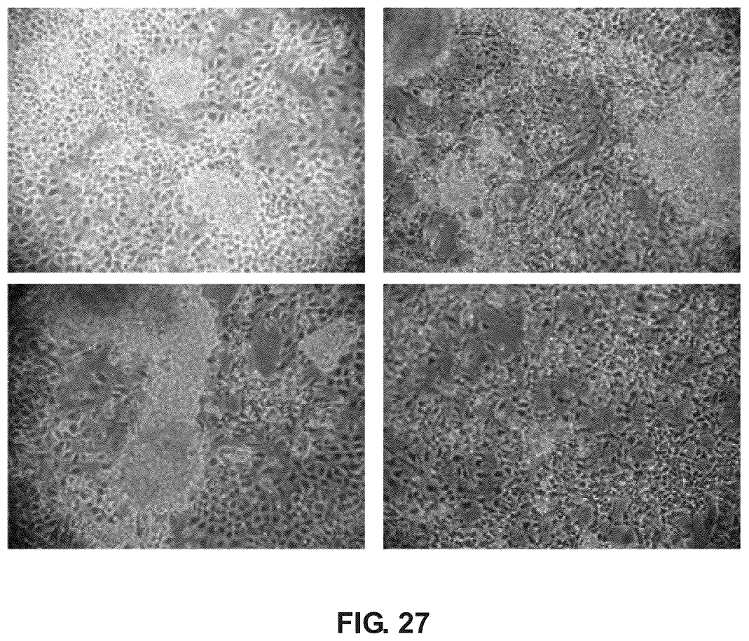

FIG. 22 shows NT-ESC colonies on MEFs before splitting (100× magnification). FIG. 23 shows NT-ESCs on gelatin immediately before starting differentiation (100× magnification). FIG. 24 shows cells on day 2 of differentiation (100× magnification) before the differentiation media was changed from 5 μM CHIR to 2 μM CHIR. CHIR causes mesodermal differentiation. Non-mesodermal cells formed clumps and can be seen floating. FIG. 25 shows cells on day 4 of EC differentiation (100× magnification) before the media was changed. EC colonies appear below the floating, undifferentiated cell clusters. FIG. 26 shows EC cells on day 7 of differentiation (100× magnification). EC colonies were visible and have become more confluent. Only a few undifferentiated cell clusters were visible. FIG. 27 shows cells after day 12-day 14 and prior to MACS sorting (100× magnification).

### Example 10-HIP Cells Differentiated into Murine Endothelial Cells (miECs)

HIP iPSC and miPSC were differentiated into Endothelial cells (miEC) but only the HIP cell-derived miECs survived long term in an allogeneic host. HIP and miPSC were plated on gelatin in 6-well plates and maintained in mouse iPSC media. After the cells reached 60% confluency, the differentiation was started and media was changed to RPMI-1640 containing 2% B-27 minus Insulin (both Gibco) and 5 μM CHIR-99021 (Selleckchem, Munich, Germany). On day 2, the media was changed to reduced media: RPMI-1640) containing 2% B-27 minus Insulin (both Gibco) and 2 μM CHIR-99021 (Selleckchem). From day 4 to day 7, cells were exposed to RPMI-1640 EC media. RPMI-1640) containing 2% B-27 minus Insulin plus 50 ng/ml mouse vascular endothelial growth factor (mVEGF; R&D Systems, Minneapolis, MN), 10 ng/mL mouse fibroblast growth factor basic (mFGFb; R&D Systems), 10 μM Y-27632 (Sigma-Aldrich, Saint Louis, MO), and 1 μM SB 431542 (Sigma-Aldrich). Endothelial cell clusters were visible from day 7 and cells were maintained in EGM-2 SingleQuots media (Lonza) plus 10% FCS hi (Gibco), 25 ng/ml mVEGF, 2 ng/mL mFGFb, 10 μM Y-27632 (Sigma-Aldrich), and 1 μM SB 431542. The differentiation process was completed after 21 days and undifferentiated cells detached during the differentiation process. For purification, cells went through MACS purification according the manufactures' protocol using anti-CD15 mAb-coated magnetic microbeads (Miltenyi, Auburn, CA) for negative selection.

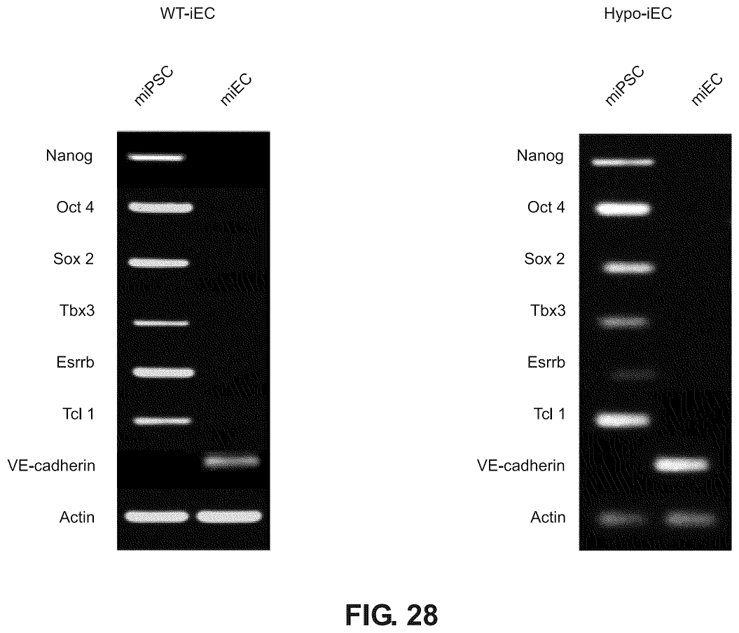

Endothelial cell differentiation was confirmed by rtPCR) and immunofluorescence. The highly purified HIP and miECs from the flow-through were cultured in EGM-2 SingleQuots media plus supplements and 10% FCS hi. TrypLE was used for passaging the cells 1:3 every 3 to 4 days. PCR was performed as described above. The following primers were used: VE-Cadherin forward primer (SEQ ID NO:13): 5′-GGATGCAGAGGCTCACAGAG-3′, and the reverse primer (SEQ ID NO:14): 5′-CTGGCGGTTCACGTTGGACT-3′. The EC cells from both the miPSCs and the HIP cells showed a differentiated gene expression profile, including VE-cadherin expression, where the parent cells did not (FIG. 28).

Their phenotype was also confirmed by immunofluorescence (IF) for CD31 (ab28364, Abcam), and VE-Cadherin (sc-6458, Santa Cruz Biotechnology, Santa Cruz, CA). Briefly, cells were fixed with 4% paraformaldehyde in PBS for 15 min. Cell membranes were permeabilized with Permeabilization solution (ASB-0102, Applied StemCell), followed by Blocking solution (ASB-0103, Applied StemCell) and incubation with the primary antibodies. For visualization, cells were incubated with secondary antibody conjugated with AF488 or AF555 (Invitrogen). After nuclei staining with DAPI, images were obtained and analyzed with a Leica SP5 laser confocal microscope (Leica). The EC cells from both the miPSCs and the HIP cells both stained positively for CD31 and VE-Cadherin (data not shown).

Tube formation was also confirmed by an immunofluorescent assay. 2.5×10⁵ miECs were stained with 5 μM CFSE and 0.1 μg/mL Hoechst (both Thermo Fisher) for 10 minutes at room temperature and plated onto 10 mg/mL undiluted Matrigel (356231, Corning, Corning, NY) in 24-well plates. After 48 h, tube formations were visualized by IF (data not shown).

### Example 6-mHIP Cell-Derived Endothelial Cells Survive Allogeneic Transplantation

Allogeneic hypo-mECs survived in an in vivo in a hindlimb ischemia model. Grafts of Fluc+wt or B2M−/−CIITA−/−CD47 tg mECs were transplanted into allogeneic mice after removal of the A. femoralis and were longitudinally followed by BLI. Fifteen animals were used per group

For bioluminescence imaging (BLI), D-luciferin firefly potassium salt (375 mg/kg: Biosynth AG, Staad, Switzerland) was dissolved in PBS (pH 7.4) (Gibco, Invitrogen) and was injected intraperitoneally (250 μL per mouse) into anesthetized mice. Animals were imaged using the IVIS 200 system (Xenogen). Region of interest (ROI) bioluminescence was quantified in units of maximum photons per second per centimeter square per steradian (p/s/cm2/sr). The maximum signal from an ROI was measured using Living Image software (MediaCybernetics). Mice were monitored on day 0, day 1, and every other day until day 30 and every 10 days afterwards.

The BLI values of all animals were plotted. Very immunogenic wt mECs were rejected within 15 days showing declining BLI signals over time while the B2M−/−CIITA−/−CD47 tg grafts all survived (FIG. 29).

### Example 11—mHIP Cell-Derived EC Cells Evaded Immune Responses In Vitro

The HIP cell-derived endothelial cells did not evoke IFN-γ or natural killer responses in vitro.

Elispot assays. For uni-directional Enzyme-Linked ImmunoSpot (Elispot) assays, recipient splenocytes were isolated from spleens 5 days after cell injection and used as responder cells. Donor cells were mitomycin-treated (50 μg/mL for 30 min.) and used as stimulator cells. One hundred thousand stimulator cells were incubated with 1×106 recipient responder splenocytes for 24 h and IFN-γ and IL-4 spot frequencies were enumerated using an Elispot plate reader.

Donor-specific antibodies. Sera from recipient mice were de-complemented by heating to 56° C. for 30 min. Equal amounts of sera and cell suspensions (5×106/mL) were incubated for 45 min at 4° C. Cells were labeled with FITC-conjugated goat anti-mouse IgM (Sigma-Aldrich) and analyzed by flow cytometry (BD Bioscience).

Mouse NK cell Elispot assays in vitro. NK cells were isolated from fresh BALB/c spleens 18 h after poly I: C injection (150 ng Poly I: C in 200 μL sterile saline, intraperitoneally (i.p.), Sigma-Aldrich). After red cell lysis, cells were purified by anti-CD49b mAb-coated magnetic bead-sorting and were used as responder cells. This cell population was >99% CD3− and contained NK cells (>90%) and other cells including myeloid cells (<10%). Using the Elispot principle, NK cells were co-cultured with B2m−/−Ciita−/− or B2m−/−Ciita−/−Cd47 tg miPSCs in the presence of IL-2 (Ing/mL, Peprotech, Rocky Hill, NJ) and their IFN-γ release was measured. YAC-1 cells (Sigma-Aldrich) served as a positive control. Mitomycin-treated (50 μg/mL for 30 min.) stimulator cells were incubated with NK cells (1:1) for 24 h and IFN-γ spot frequencies were enumerated using an Elispot plate reader.

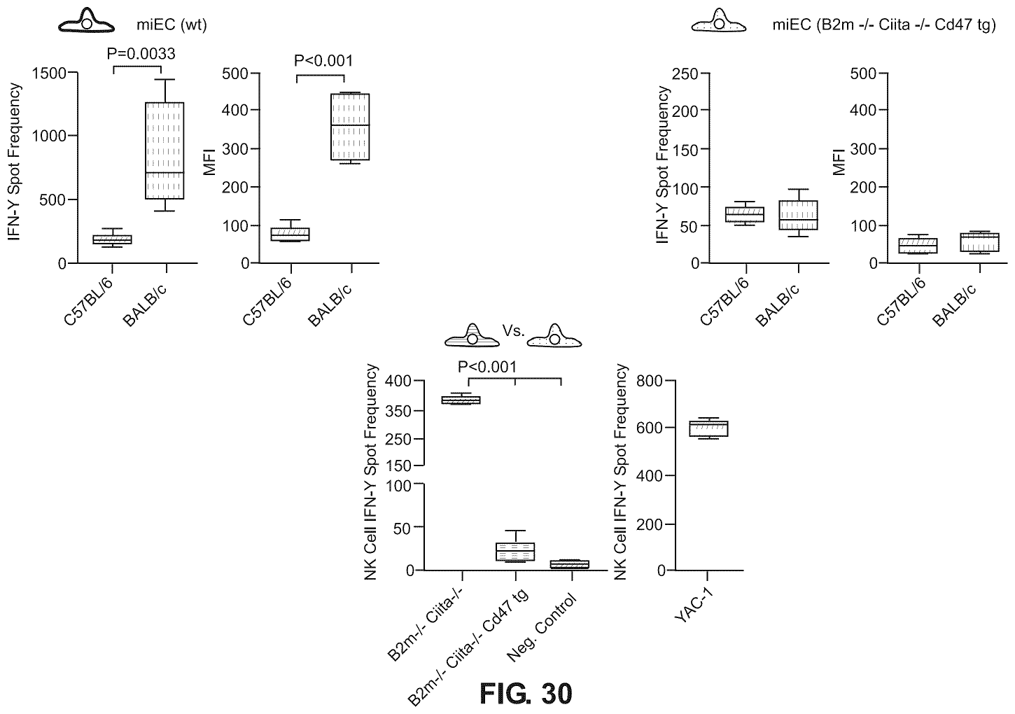

Five days after the injection of wt miPSC-derived miECs into C57BL/6 or BALB/c recipients, splenocytes were recovered for IFN-γ Elispot assays (box 25th to 75th percentile with median, whiskers min-max, 6 animals per group, two-tailed Student's t-test). The IFN-γ response was vastly stronger in all allogeneic recipients. Mean fluorescence (MFI) of IgM binding to wt miPSC-derived miECs incubated with recipient serum after 5 days (box 25th to 75th percentile with median, whiskers min-max, 6 animals per group, two-tailed Student's t-test). There was a markedly stronger IgM response in all allogeneic recipients (FIG. 30).

Similarly, B2m^{-/-}Ciita^{-/-} Cd47 tg miPSC-derived miECs were injected into C57BL/6 or BALB/c recipients and IFN-γ Elispots were performed after 5 days (box 25th to 75th percentile with median, whiskers min-max, 6 animals per group, two-tailed Student's t-test). Mean fluorescence (MFI) of IgM binding to B2m^{-/-}Ciita^{-/-} Cd47 tg miPSC-derived miEC), incubated with recipient serum after 5 days (box 25th to 75th percentile with median, whiskers min-max, 6 animals per group, two-tailed Student's t-test). There was no measurable IFN-γ response or IgM response in allogeneic recipients. To assess the inhibitory effect of Cd47 over-expression on NK cell killing, IFN-γ Elispots with NK cells were performed with miECs derived from B2m^{-/-}Ciita^{-/-} miPSC or B2m^{-/-}Ciita^{-/-} Cd47 tg miPSC (box 25th to 75th percentile with median, whiskers min-max, 6 independent experiments, ANOVA with Bonferroni's post-hoc test) Only derivatives from B2m Ciita-miPSC were susceptible for NK cell killing (FIG. 30).

### Example 12-HIP Cell-Derived Endothelial Cell Morphology

HIP cell-derived endothelial cells showed the typical EC morphology. B2m^{-/-}Ciita^{-/-} Cd47 tg miEC grafts in matrigel were transplanted subcutaneously into allogeneic BALB/c mice. These hypo-immunogenic derivatives further matured in vivo or changed their morphology over time in allogeneic recipients.

Eight hundred thousand B2m−/−Ciita−/−Cd47 tg miECs in 1:1 diluted Matrigel (Corning) were injected into allogeneic BALB/c mice. Matrigel plugs were recovered at multiple time points up to 56 days and fixed in 4% paraformaldehyde in PBS with 1% glutaraldehyde for 24 h. Samples were dehydrated, embedded in paraffin, and cut into sections of 5 μm thickness. For histopathology, sections were stained with hematoxylin and eosin (Carl Roth) and images taken with an inverted light microscope. The origin of the cells was demonstrated with immunofluorescence staining. Sections were rehydrated and underwent antigen-retrieval and blocking. Samples were incubated with antibodies against luciferase (ab21176), VE-Cadherin (SC-6458) and a corresponding secondary antibody conjugated with AF488 or AF555 (Invitrogen). Cell nuclei were counterstained with DAPI and images taken with a Leica SP5 laser confocal microscope (Leica, Wetzlar, Germany).

For co-staining experiments of miECs and immune cells, primary antibodies were used against VE-Cadherin (SC-6458, Sigma) and CD3 (ab16669, Abcam), followed by the corresponding secondary antibody conjugated with AF488 or AF555 (Invitrogen). Premature vessel formation was observed. Data not shown.

Transplanted miECs started to organize in circular structures around day 14 and formed primitive vessels that contained erythrocytes around week 3, (Data not shown).

Perfusion doppler (Periscan PIM II″ (PERIMED Ltd., Italy) of the cells taken from the animals from Example 6 demonstrated new vessel formation and rescued the limb in the hypo-EC group (Data Not Shown).

### Example 13-Human HIP Cells Differentiated into Endothelial Cells

Human HIP cells were differentiated into hiEC cells. Wild-type hiPSC and human HIP cells were plated on diluted Matrigel (356231, Corning) in 6-well plates and maintained in Essential 8 Flex media (Thermo Fisher). The differentiation was started at 60% confluency, and media was changed to RPMI-1640 containing 2% B-27 minus insulin (both Gibco) and 5 μM CHIR-99021 (Selleckchem). On day 2, the media was changed to reduced media: RPMI-1640 containing 2% B-27 minus insulin (Gibco) and 2 μM CHIR-99021 (Selleckchem). From day 4 to day 7, cells were exposed to RPMI-1640 EC media, RPMI-1640 containing 2% B-27 minus insulin plus 50 ng/ml human vascular endothelial growth factor (VEGF; R&D Systems), 10 ng/ml human fibroblast growth factor basic (FGFb; R&D Systems), 10 μM Y-27632 (Sigma-Aldrich), and 1 μM SB 431542 (Sigma-Aldrich). Endothelial cell clusters were visible from day 7 and cells were maintained in EGM-2 SingleQuots media (Lonza) plus 10% FCS hi (Gibco), 25 ng/mL VEGF, 2 ng/ml FGFb, 10) μM Y-27632 (Sigma-Aldrich), and 1 μM SB 431542 (Sigma-Aldrich). The differentiation process was completed after 14 days und undifferentiated cells detached during the differentiation process. For purification, cells were treated with 20 μM PluriSln-1 (StemCell Technologies, Vancouver, BC, Canada) for 48 h. The highly purified ECs were cultured in EGM-2 SingleQuots media (Lonza) plus supplements and 10% FCS hi (Gibco). TrypLE Express was used for passaging the cells 1:3 every 3 to 4 days.

IF staining was performed as described above to confirm their phenotype. Primary antibodies were used against CD31 (ab28364, Abcam) and VE-Cadherin (sc-6458, Santa Cruz Biotechnology), followed by the corresponding secondary antibody conjugated with AF488 or AF555 (Invitrogen). Cell nuclei were stained with DAPI. Imaging was performed using a Leica SP5 laser confocal microscope (Leica). PCR for VE-Cadherin (forward (SEQ ID NO: 15): 5′-AAGATGCAGAGGCTCATG-3′, and the reverse primer (SEQ ID NO:16): 5′-CATGAGCCTCTGCATCTT-3′) was performed as described above.

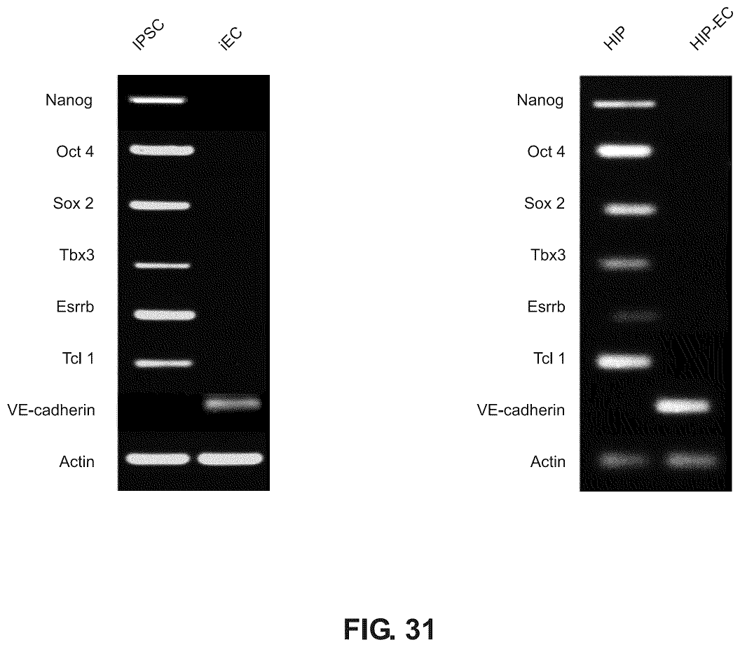

wt hiPSCs (a) and B2M-CHITA-CD47 tg hiPSCs (b) were successfully differentiated into corresponding hiEC derivatives. The EC cells from both the hiPSCs and the HIP cells showed a differentiated gene expression profile, including CDH5 expression, where the parent cells did not. miECs were positive for CD31 and VE-cadherin by confocal immunofluorescence. All derivatives lost their expression of pluripotency genes (representative pictures of two independent experiments) (FIG. 31 and data not shown).

### Example 14-hiEC Cells Survived in Humanized Mice

Human HIP cells survived grafting into mice with humanized immune systems. Humanized NSG-SGM3 mice (18-30 weeks) were purchased from Jackson Laboratories (Cat. No. SMG3-CD34, Sacramento, CA). Human CD34+ hematopoietic stem cell (HSE)-engrafted NSG-SGM3 mice develop multi-lineage human immune cells. They demonstrate a functional human immune system displaying T cell-dependent immune responses with no donor cell immune reactivity towards the host. Animals were randomly assigned to experimental groups. The percentage of CD3+ cells among the human CD45+ cell population was assessed in every animal and CD3 percentages were never significantly different between wild-type and B2M−/−CIITA−/−CD47 tg groups. All humanized NSG-SGM3 mice were HLA-A typed and the number of mismatches to the cell graft calculated. In Elispot assays with hiEC groups there were always 2 mismatches.

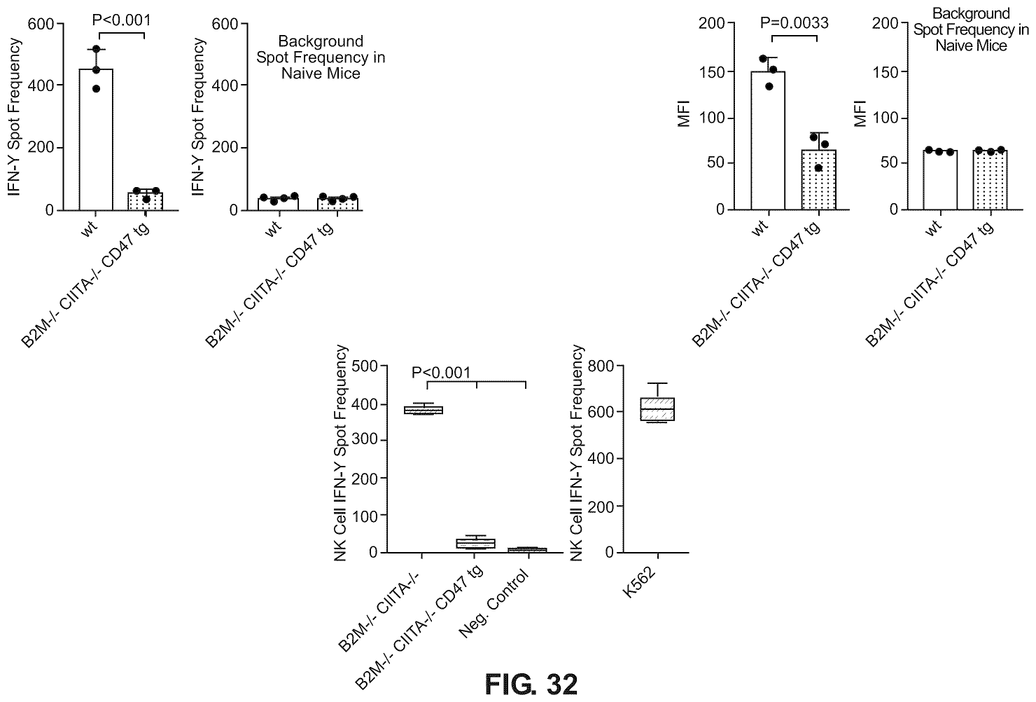

Wild-type or B2M−/−CIITA−/−CD47 tg hiEC grafts were injected into allogeneic humanized NSG-SGM3 mice. IFN-γ Elispots were performed after 5 days (mean±s.d., 3 animals per group, two-tailed Student's t-test). The background spot frequency in naive mice is shown (mean±s.d., 4 animals per group, two-tailed Student's t-test). 1. MFI of IgM binding to either hiEC incubated with recipient serum after 5 days (mean±s.d., 3 animals per group, two-tailed Student's t-test). The background fluorescence in naive mice is shown (mean±s.d., 3 animals per group, Student's t-test). IFN-γ Elispots with human NK cells were performed with B2M−/−CIITA−/−hiECs or B2M−/−CIITA−/−CD47 tg hiECs (box 25th to 75th percentile with median, whiskers min-max, 6 independent experiments, ANOVA with Bonferroni's post-hoc test). (FIG. 32.)

All publications and patent documents disclosed or referred to herein are incorporated by reference in their entirety. The foregoing description has been presented only for purposes of illustration and description. This description is not intended to limit the invention to the precise form disclosed.

It is intended that the scope of the invention be defined by the claims appended hereto.

1.-164. (canceled)

165. An isolated population of hypoimmune cardiac cells differentiated from a hypoimmune pluripotent cell (HIP cell),
wherein an endogenous β-2 microglobulin (B2M) gene activity and an endogenous class II transactivator (CIITA) gene activity have been eliminated, wherein a CD47 expression has been increased, and wherein more than 50% of hypoimmune cardiac cell areas in a cell culture beat after 10 days of said differentiation.

166. The isolated population of hypoimmune cardiac cells of claim 165, wherein said HIP cell is an induced pluripotent stem cell (iPSC) or an embryonic stem cell.

167. The isolated population of hypoimmune cardiac cells of claim 166, wherein said HIP cell is a human iPSC, wherein said increased CD47 expression results from an exogenous human CD47 gene under the control of a promoter within said hypoimmune cardiac cell.

168. The isolated population of hypoimmune cardiac cells of claim 165, wherein said elimination of B2M gene activity results from a disruption of both alleles of the B2M gene.

169. The isolated population of hypoimmune cardiac cells of claim 165, wherein said elimination of CIITA gene activity results from a disruption of both alleles of the CIITA gene.

170. The isolated population of hypoimmune cardiac cells of claim 165, further comprising a suicide gene that is activated by a trigger agent, wherein a triggered expression of said suicide gene induces said hypoimmune cardiac cell to die.

171. The isolated population of hypoimmune cardiac cells of claim 170, wherein said suicide gene is a herpes simplex virus thymidine kinase (HSV-tk) gene and said trigger agent is ganciclovir.

172. The isolated population of hypoimmune cardiac cells of claim 170, wherein said suicide gene is an Escherichia coli cytosine deaminase (CD) gene and said trigger agent is 5-fluorocytosine (5-FC).

173. The isolated population of hypoimmune cardiac cells of claim 170, wherein said suicide gene encodes an inducible caspase 9 protein and said trigger agent is a chemical inducer of dimerization (CID).

174. The isolated population of hypoimmune cardiac cells of claim 165, wherein said isolated population of hypoimmune cardiac cells comprise cardiomyocytes, nodal cardiomyocytes, conducting cardiomyocytes, working cardiomyocytes, cardiomyocyte precursors, cardiomyocyte progenitor cells, cardiac stem cells, or cardiac muscle cells.

175. A composition, comprising the population of isolated hypoimmune cardiac cells of claim 165 and a pharmaceutical carrier.

176. A method of treating a heart condition or disease, comprising administering a therapeutically effective amount of the composition of claim 175 to a patient in need thereof.

177. The method of claim 176, wherein said composition is administered via implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

178. The method of claim 176, wherein said heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

179. A method of producing the isolated population of hypoimmune cardiac cells of claim 165, comprising differentiating a hypoimmune pluripotent cell (HIP cell) in vitro into said isolated population of hypoimmune cardiac cells, said method comprising:
(a) culturing said population of HIP cells in a culture medium comprising a GSK inhibitor to produce a first population of cells;
(b) culturing said first population of cells in a culture medium comprising a WNT antagonist to produce a population of pre-cardiac cells; and
(c) culturing said population of pre-cardiac cells in a culture medium comprising insulin to produce a population of cardiac cells,
wherein said cardiac cells are beating cardiac cells.

180. The method of claim 179, wherein said GSK inhibitor is CHIR-99021, a derivative thereof, or a variant thereof.

181. The method of claim 179, wherein said WNT antagonist is IWR1,
a derivative thereof, or a variant thereof.

182. The method of claim 179, wherein said HIP cells further comprise a suicide gene.

183. The method of claim 179, further comprising isolating said population of hypoimmune cardiac cells away from non-cardiac cells.

184. A cryopreserved composition comprising the composition of claim 175.


SEQUENCE LISTING (16 sequences)

SEQ ID NO: 1 - protein, 119 aa, Homo sapiens [sequence omitted]

SEQ ID NO: 2 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 3 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 4 - protein, 376 aa, Herpes simplex virus
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 6 - protein, 282 aa, Homo sapiens
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 7 - DNA, 20 bp, synthetic construct
  misc_feature 1..20: note = Description of Artificial Sequence: Synthetic primer
  source 1..20: mol_type = other DNA
ctgtcatctcactatgggca

SEQ ID NO: 8 - DNA, 20 bp, synthetic construct
  misc_feature 1..20: note = Description of Artificial Sequence: Synthetic primer
  source 1..20: mol_type = other DNA
ccaagtccgagcaggaattt

SEQ ID NO: 9 - DNA, 21 bp, synthetic construct
  misc_feature 1..21: note = Description of Artificial Sequence: Synthetic primer
  source 1..21: mol_type = other DNA
atcattcccaacgagcgaaag

SEQ ID NO: 10 - DNA, 21 bp, synthetic construct
  misc_feature 1..21: note = Description of Artificial Sequence: Synthetic primer
  source 1..21: mol_type = other DNA
aagtccccatagagaatgcgg

SEQ ID NO: 11 - DNA, 25 bp, synthetic construct
  misc_feature 1..25: note = Description of Artificial Sequence: Synthetic primer
  source 1..25: mol_type = other DNA
gaggcaccaagttgggcatgaacga

SEQ ID NO: 12 - DNA, 22 bp, synthetic construct
  misc_feature 1..22: note = Description of Artificial Sequence: Synthetic primer
  source 1..22: mol_type = other DNA
ggcagcggaagaggatgctgaa

SEQ ID NO: 13 - DNA, 20 bp, synthetic construct
  misc_feature 1..20: note = Description of Artificial Sequence: Synthetic primer
  source 1..20: mol_type = other DNA
ggatgcagaggctcacagag

SEQ ID NO: 14 - DNA, 20 bp, synthetic construct
  misc_feature 1..20: note = Description of Artificial Sequence: Synthetic primer
  source 1..20: mol_type = other DNA
ctggcggttcacgttggact

SEQ ID NO: 15 - DNA, 18 bp, synthetic construct
  misc_feature 1..18: note = Description of Artificial Sequence: Synthetic primer
  source 1..18: mol_type = other DNA
aagatgcagaggctcatg

SEQ ID NO: 16 - DNA, 18 bp, synthetic construct
  misc_feature 1..18: note = Description of Artificial Sequence: Synthetic primer
  source 1..18: mol_type = other DNA
catgagcctctgcatctt